(19) United States
(12) Patent Application Publication
(10) Pub. No.: US 2026/0284079 A1
(43) Pub. Date: Sep. 24, 2026

(54) LIPID PRODRUGS OF ALLOPREGNANOLONE AND USES THEREOF

(71) Applicant: Seaport Therapeutics, Inc., Seaport Boston, MA (US)

(72) Inventors: Michael C. Chen, Norwood, MA (US); Daniel Kenneth Bonner, Sharon, MA (US); James E. Shipley, Cary, NC (US); Janet M. Schnee, Newtown, CT (US); Eric Elenko, Boston, MA (US)

(73) Assignee: Seaport Therapeutics, Inc., Seaport Boston, MA (US)

(21) Appl. No.: 19/675,377

(22) Filed: May 12, 2026

Related U.S. Application Data
(63) Continuation of application No. PCT/US2024/055488, filed on Nov. 12, 2024.
(60) Provisional application No. 63/598,495, filed on Nov. 13, 2023.
(60) Provisional application No. 63/561,060, filed on Mar. 4, 2024.
(60) Provisional application No. 63/635,832, filed on Apr. 18, 2024.

(51) Int. Cl. A61K 31/573; A61P 25/22; A61P 25/24

(52) U.S. Cl. CPC A61K 31/573; A61P 25/22; A61P 25/24

(57) ABSTRACT

Disclosed herein are methods of treating a subject in need thereof with an orally-administered lipid prodrug of allopregnanolone. The methods described herein may be used for the treatment of major depressive disorder (MDD) with anxious distress or major depressive disorder (MDD) without anxious distress.

### CROSS-REFERENCE TO RELATED APPLICATIONS

This application is a continuation of International Application No. PCT/US2024/055488 filed on Nov. 12, 2024, which claims the benefit of priority to U.S. Provisional Application No. 63/598,495, filed on Nov. 13, 2023, U.S. Provisional Application No. 63/561,060, filed on Mar. 4, 2024, and U.S. Provisional Application No. 63/635,832, filed on Apr. 18, 2024, the entireties of which are incorporated herein by reference.

### FIELD OF INVENTION

The present disclosure relates generally to the oral administration of lipid prodrugs of allopregnanolone for the treatment of a human subject in need thereof.

### SUMMARY OF INVENTION

In one aspect disclosed herein is a method of treating a human subject, the method comprising orally administering to the human subject in need thereof a dose of Compound 1:

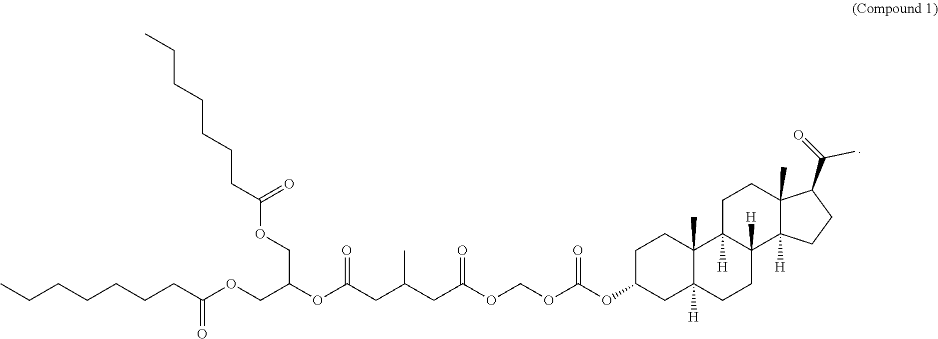

In some embodiments, the dose of Compound 1 is between 70-500 mg. In some embodiments, the dose of Compound 1 is 70 mg, 125 mg, 140 mg, 250 mg, 280 mg, 375 mg, 420 mg, or 500 mg. In some embodiments, the dose of Compound 1 is 125 mg. In some embodiments, the dose of Compound 1 is 250 mg. In some embodiments, the dose of Compound 1 is 375 mg.

In some embodiments, the human subject in need thereof has major depressive disorder (MDD). In some embodiments, the human subject has MDD with anxious distress. In some embodiments, the human subject has MDD without anxious distress. In some embodiments, the dose of Compound 1 is administered once per day. In some embodiments, the dose of Compound 1 is administered once per day at bedtime (qHS).

In one aspect, provided herein is a method of treating a human subject having major depressive disorder (MDD), the method comprising orally administering to the human subject in need thereof: i) a daily initiation dose of Compound 1 for an initiation period of up to 7 days; and ii) a daily maintenance dose of Compound 1 that begins after the initiation period, wherein Compound 1 is:

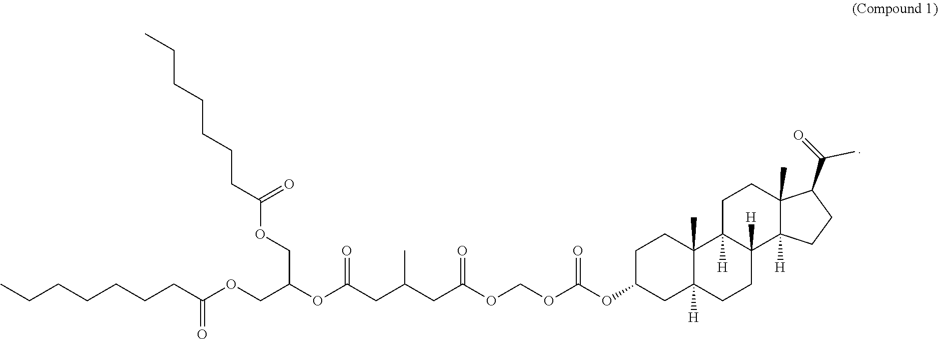

In some embodiments, the daily initiation dose of Compound 1 is 125 mg. In some embodiments, the daily initiation dose of Compound 1 is 250 mg. In some embodiments, the daily maintenance dose of Compound 1 is 125 mg, 250 mg, or 375 mg. In some embodiments, the initiation period is 3 to 7 days. In some embodiments, the daily maintenance dose of Compound 1 is greater than the daily initiation dose of Compound 1. In some embodiments, the daily maintenance dose of Compound 1 is the same as the daily initiation dose of Compound 1. In some embodiments, the daily maintenance dose of Compound 1 is smaller than the daily initiation dose of Compound 1.

### BRIEF DESCRIPTION OF THE FIGURES

FIGS. 1A-1C graphically depict PK parameters determined from the single ascending dose study (Part 1 of Example 1). FIG. 1A depicts the dose-exposure relationship of AUC_{0-24 h}.

FIG. 1B depicts the dose-exposure relationship of Cmax. FIG. 1C depicts the concentration of allopregnanolone over time for the tested doses.

FIGS. 2A-2C graphically depict PK parameters determined from the multiple ascending dose study (Part 3 of Example 1). FIG. 2A depicts plasma concentrations of allopregnanolone in subjects dosed at 250 mg QAM (Cohort 4), 375 mg QAM (Cohort 5), 375 mg QHS (Cohort 8), and 500 mg QAM (Cohort 6). FIG. 2B depicts the dose-exposure relationship of AUC_{0-24 h} at day 7. FIG. 2C depicts the dose-exposure relationship of Cmax at day 7.

FIGS. 3A-3B depict the PD assessments from the multiple ascending dose study (Part 3 of Example 1). FIG. 3A depicts the change in saccadic eye velocity in subjects dosed at 250 mg (Cohort 4), 375 mg (Cohort 5), and 500 mg (Cohort 6) of Compound 1 with eyes open (electrode Cz). FIG. 3B depicts the change in beta power in subjects dosed at 250 mg (Cohort 4), 375 mg (Cohort 5), and 500 mg (Cohort 6) of Compound 1. Change in saccadic eye velocity (FIG. 3A) and beta power (FIG. 3B) are expressed as a ratio of the value at a given timepoint to the pre-dose value (time=0 hrs).

FIGS. 4A-4C graphically depict the change in salivary cortisol levels in subjects treated with Compound 1 or placebo. FIG. 4A shows the change from baseline in salivary cortisol levels over time. FIG. 4B shows the maximum change from baseline. FIG. 4C depicts the study design described in Example 2.

### DETAILED DESCRIPTION

Disclosed herein are methods for treating a subject in need thereof with a lipid prodrug of allopregnanolone.

### Definitions

While the terms used herein are believed to be well understood by one of ordinary skill in the art, certain definitions are set forth herein to facilitate explanation of the present disclosure.

As used herein, the term “Adverse Event” refers to any event, side-effect, or other untoward medical occurrence in a subject (i.e., a clinical study participant) administered a pharmaceutical product. An adverse event does not necessarily have a causal relationship with the pharmaceutical product. As used herein, a “treatment-emergent adverse event” or “TEAE” refers to an adverse event not present prior to treatment (i.e., receiving a dose of Compound 1) or an already present event that worsens in intensity or frequency following treatment. TEAEs may be classified by system organ class (SOC); an exemplary SOC is Nervous System Disorders. Examples of Nervous System Disorder TEAEs include somnolence, headache, dizziness, lethargy, balance disorder, and cognitive disorder.

As used herein, the term “treatment”, “treat”, and “treating” refer to reversing, alleviating, preventing, mitigating, delaying the onset of, or inhibiting the progression of a disorder, or one or more symptoms thereof. For example, when these terms are used with respect to treatment of anxiety, “treating anxiety” includes both alleviating existing anxiety or preventing anxiety, as well as managing anxiety. The anxiety disorder can include generalized anxiety disorder, obsessive compulsive disorder, panic disorder, social anxiety disorder, or any of the anxiety disorders disclosed herein. Treating can also include treating both depression and anxiety.

The terms “subject”, “patient”, and “participant” refer to a mammalian subject, including a human subject. In some embodiments, the subject is a human subject.

As used herein, the term “anxiety disorder” refers to one or more mental disorders characterized by feelings of worry, anxiety, fear, and stress, amongst others. Anxiety disorders may include, but are not limited to, social anxiety disorder and generalized anxiety disorder (GAD).

As used herein, the term “stress” is the result of a feeling or thought, either conscious or unconscious, resulting from a traumatic event or external pressure (e.g., a stressor). As used herein, the term “stress-related disorder” refers to disorders that develop as a result of stress. In some instances, the stress-related disorder may have symptoms that overlap with one or more anxiety disorders. In some instances, anxiety disorders (e.g., social anxiety disorder or generalized anxiety disorder, GAD) may be originated, induced, or exacerbated by stress. Stress can be acute, episodic, or chronic. A human subject may suffer from one or more stressors, or one or more stress-related disorders, and may exhibit one or more symptoms associated with the stressors and/or stress-related disorders thereof. Stress-related disorders (including stress-related or stress-induced anxiety disorders) include, but are not limited to, separation anxiety disorder, selective mutism, specific phobias, social phobias, panic disorder (e.g., nocturnal panic disorder and/or panic attacks), agoraphobia, generalized anxiety disorder, substance/medication-induced anxiety disorder, anxiety disorders due to another medical condition, other specified anxiety disorders, unspecified anxiety disorders, reactive attachment disorder, disinhibited social engagement disorder, acute stress disorder, adjustment disorder, post-traumatic stress disorder, prolonged grief disorder, social anxiety disorder, other specified trauma- and stressor-related disorders, or unspecified trauma- and stressor-related disorders. A human subject in need thereof may have a depression or mood disorder, including, but not limited, to major depressive disorder (MDD), bipolar disorder, seasonal affective disorder, cyclothymic disorder, premenstrual dysphoric disorder, persistent depressive disorder, disruptive mood dysregulation disorder, postpartum depression, perimenopausal depression, or a depression related to a medical illness. A human subject in need thereof may have an anxiety disorder and/or stress-related disorder concurrent with one or more depression or mood disorders or symptoms of a depression or mood disorder. For example, a subject in need thereof may have generalized anxiety disorder (GAD) with one or more depression or mood disorders or one or more symptoms of a mood or depression disorder such as, but not limited to, major depressive disorder. In some embodiments, a subject in need thereof may have major depressive disorder (MDD) with anxiety (also referred to as MDD with anxious distress).

As used herein, “MDD with anxiety” or “MDD with anxious distress” refers to the presence of at least two of the symptoms described below during the majority of days of the current major depressive disorder episode, past major depressive disorder episode, or a current depressive disorder episode. Symptoms of anxious distress include i) feeling keyed up or tense, ii) feeling unusually restless, iii) difficulty concentrating because of worry, iv) fear that something awful may happen, and/or v) feeling that the individual might lose control of themself. Severity of MDD with anxious distress can further be classified as mild (2 of the above symptoms), moderate (3 of the above symptoms), moderate-severe (4-5 of the above symptoms), or severe (4-5 of the above symptoms plus motor agitation).

As used herein, the term “daily initiation dose” refers to a daily dose of a compound of Formula I, e.g., Compound 1, that is administered during an initial period of up to and including 7 days. In some embodiments, the initial period is 3 days, 4 days, 5 days, 6 days, or 7 days. In some embodiments, the daily initiation dose is 125 mg of Compound 1. In some embodiments, the daily initiation dose is 250 mg of Compound 1.

As used herein, the term “daily maintenance dose” refers to a daily dose of a compound of Formula I, e.g., Compound 1, that is administered following the initial period of up to and including 7 days. The daily maintenance dose may be the same, higher, or lower than the daily initiation dose. In some embodiments, the daily maintenance dose is 125 mg of Compound 1. In some embodiments, the daily maintenance dose is 250 mg of Compound 1. In some embodiments, the daily maintenance dose is 375 mg of Compound 1. The daily maintenance dose is determined based on both the observed efficacy and tolerability in a given subject at a particular dose amount. The daily maintenance dose may stay the same or may be increased or decreased based on the observed efficacy and observed tolerability in a given subject at said dose amount. For example, but not by way of limitation, observed efficacy may be determined based on a change from baseline in the Hamilton Depression Rating Scale-17 (HAM-D-17) total score for a particular subject.

As used herein, the term “pharmaceutically acceptable carrier”, “pharmaceutically acceptable adjuvant”, or “pharmaceutically acceptable vehicle”, or combinations thereof, refer to non-toxic carriers, adjuvants, or vehicles that do not destroy the pharmacological activity of the agent with which it is formulated. Pharmaceutically acceptable carriers, adjuvants, or vehicles suitable for use in the disclosed compositions include, but are not limited to, ion exchangers, alumina, stearates, lecithins, serum proteins, buffer substances such as phosphates, glycine, sorbic acid, potassium sorbate, glyceride mixtures, lipids, water, salts, or electrolytes.

Unless otherwise stated, structures depicted herein are meant to include all isomeric (e.g., enantiomeric, diastereomeric, and geometric (or conformational) forms of the structure: for example, the R and S configurations for each asymmetric center. Therefore, single stereochemical isomers as well as enantiomeric, diastereomeric, and geometric (or conformational) mixtures are within the scope of the disclosed methods.

### Lipid Prodrugs of Allopregnanolone

In one aspect, disclosed herein are methods of treating stress-related disorders, including e.g., stress-related anxiety disorders, using a lipid prodrug of allopregnanolone (Compound 1). Allopregnanolone is an endogenous pregnane neurosteroid that acts as a positive allosteric modulator of the inhibitory action of gamma-aminobutyric acid (GABA) on the GABA type A (GABA_{A}) receptor.

GABA_{A} receptors are pentameric proteins that present in a multitude of isoforms. Typically, GABA_{A} receptors comprise two α-subunits, two β-subunits, and one γ-, δ-, ε-, π-, or θ-subunit. At least 6 α, 3 β, and 3 γ subunits have been identified to date, lending to a high number of potential pentameric GABA_{A} compositions. The abundance and distribution of GABA_{A} receptor subtypes varies widely across the central nervous system (CNS). The composition of the GABA_{A} receptor influences behavioral effects. Whereas the α₁ subunit is associated with sedation, the α₂ subunit is associated with anxiolytic effects. Additional associations between the GABA_{A} receptor subunits have been identified, for instance the α₂, α₄, β₃, and δ subunit are all known to be associated with anxiety and mood symptoms (Wang, Mingde, Front. Endrocrinol. (2011) 2:44). Thus, the mechanism of action (MOA) of a therapeutic that acts upon GABA_{A}, and its downstream pharmacodynamic effects, is dependent on both the subunit composition of the GABA_{A} receptors and the binding site of said therapeutic.

As an example, benzodiazepines act upon GABA_{A} receptors to exert anxiolytic and anti-depressant properties. The MOA depends on the binding of benzodiazepines at the interface of the α and γ subunit, thus benzodiazepines require GABA_{A} receptors that comprise a γ subunit (Goldschen-Ohm, Biomolecules (2022)). In contrast, allopregnanolone has broad specificity with respect to the GABA_{A} subunit, and further can act upon GABA_{A} receptors that contain a δ subunit. GABA_{A} receptors with δ subunits are primarily located in the extrasynaptic and perisynaptic spaces (Glkyks J, et al., J. Neurosci (2008) 28 (6): 1421-1426; Brickley and Mody (2012) Neuron 73 (1): 23-34). These GABA_{A} receptors have a high affinity for GABA and mediate tonic inhibition (Paul S et al., Neurobiology of Stress (2020)). Thus, allopregnanolone acts as a potent modulator across GABA_{A} receptor isoforms with the ability to exert antidepressant, sedative, anxiolytic, and stress-reducing effects, amongst others.

Thus, allopregnanolone has the potential to treat stress-related disorders. However, allopregnanolone suffers from low oral bioavailability, less than 5%, due to extensive first-pass metabolism. The present disclosure provides a lipid prodrug of allopregnanolone that bypasses first-pass metabolism, enabling the oral delivery of allopregnanolone. Lipid mimetic compounds, such as lipid prodrugs, can behave similarly to natural triglycerides, enabling transport through the lymphatic system prior to reaching system circulation, effectively circumventing first-pass metabolism. Lipid prodrugs are further discussed in WO2016/023082, WO2017/041139, and WO2021/159021, the entireties of which are hereby incorporated by reference.

The development of oral allopregnanolone has been restricted due to its poor oral bioavailability. An intravenous formulation of allopregnanolone (brexanolone) has been approved for the treatment of severe postpartum depression (marketed as Zulresso®), where it is administered in a hospital setting over a 60-hr infusion period. The Zulresso® infusion is titrated over the course of 60 hours (2.5 days), increasing from 30 μg/kg/hr to 60 μg/kg/hr in the first 24 hours and a continuous 90 μg/kg/hr for hours 24-52. After 52 hours of treatment, the infusion is titrated down to 60 μg/kg/hr for 4 hours and 30 μg/kg/hr for 4 hours. Patients receiving Zulresso® require continuous monitoring during this period due to the high risk of sedation and/or loss of consciousness (package insert, Zulresso®). The antidepressant efficacy following a 2.5 day infusion has been reported to last up to 30 days.

Disclosed herein is a lipid prodrug of allopregnanolone, Compound 1, that is administered orally to a subject in need thereof:

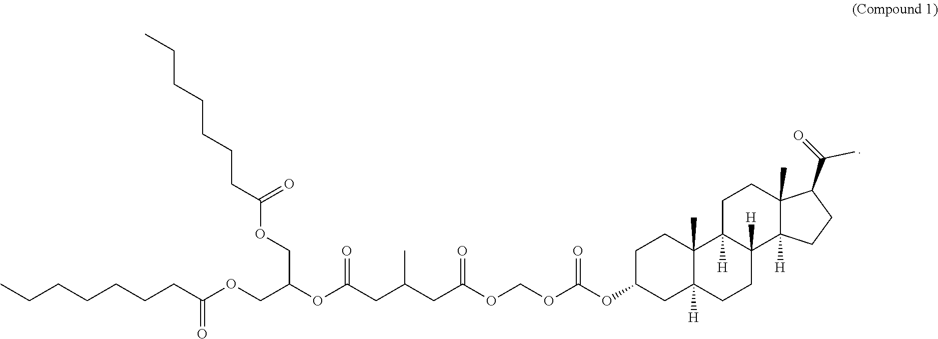

Compound 1 may be administered as a diastereomer or mixture thereof. Accordingly, in some embodiments the methods and formulations of Compound 1 disclosed herein may comprise Compound 1-R:

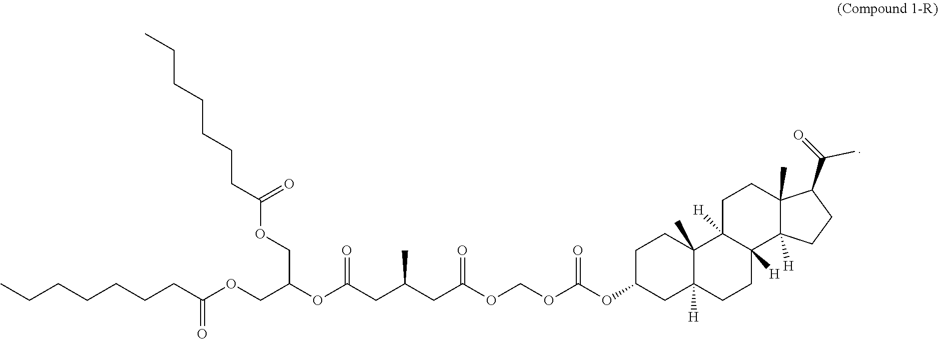 [SMILES: CCCCCCCC(=O)OCC(COC(=O)CCCCCCC)OC(=O)C[C@@H](C)CC(=O)OCOC(=O)O[C@@H]1CC[C@@]2(C)[C@@H](CC[C@@H]3[C@@H]2CC[C@]2(C)[C@@H](C(C)=O)CC[C@@H]32)C1]

In some embodiments the methods and formulations of Compound 1 disclosed herein may comprise Compound 1-S:

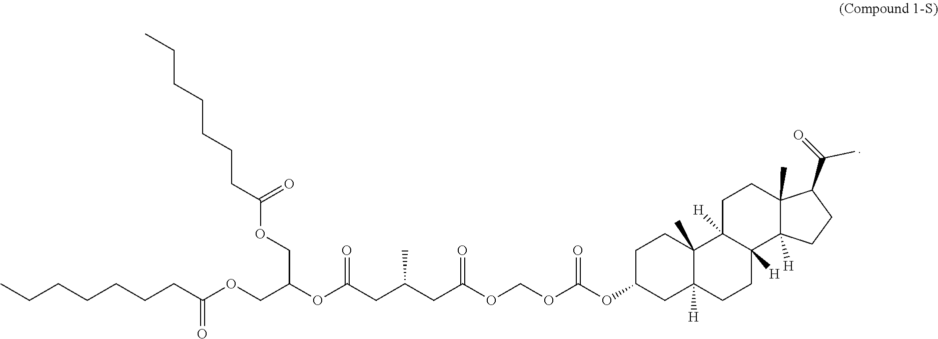 [SMILES: CCCCCCCC(=O)OCC(COC(=O)CCCCCCC)OC(=O)C[C@H](C)CC(=O)OCOC(=O)O[C@@H]1CC[C@@]2(C)[C@@H](CC[C@@H]3[C@@H]2CC[C@]2(C)[C@@H](C(C)=O)CC[C@@H]32)C1]

In some embodiments the methods and formulations of Compound 1 disclosed herein comprise a diastereomeric mixture of Compound 1-R and Compound 1-S. In some embodiments, the diastereomeric mixture of Compound 1-R and Compound 1-S is at about a 1:1 molar ratio. Compound 1 is designed to bypass first-pass metabolism and subsequently release free allopregnanolone once in systemic circulation (Lipid mimetic compounds are further discussed in WO2016/023082 and WO2017/041139, the entireties of which are hereby incorporated by reference). Compound 1 can be synthesized as described in WO2021/149021. Without wishing to be bound by any particular theory, it is believed that each diastereomer of Compound 1 (i.e., Compound 1-R and Compound 1-S) releases unmodified allopregnanolone at near equivalent levels.

In one aspect, disclosed herein is a lipid prodrug of allopregnanolone, Formula I, that is administered orally to a subject in need thereof:

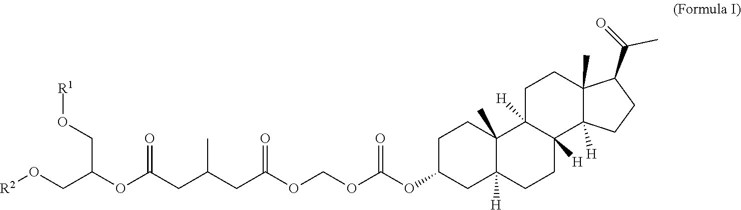

wherein R¹ and R² are each independently —C(O)R³; and
each R³ is independently a saturated or unsaturated, straight or branched, optionally substituted C₃-C₁₇ hydrocarbon chain. In some embodiments, each R³ is independently a saturated, straight C₃-C₁₇ hydrocarbon chain. In some embodiments, each R³ is independently an unsaturated, straight C₃-C₁₇ hydrocarbon chain.

A compound of Formula I may be administered as a diastereomer or mixture thereof. Accordingly, in some embodiments the methods and formulations of Formula I disclosed herein may comprise Formula I-R:

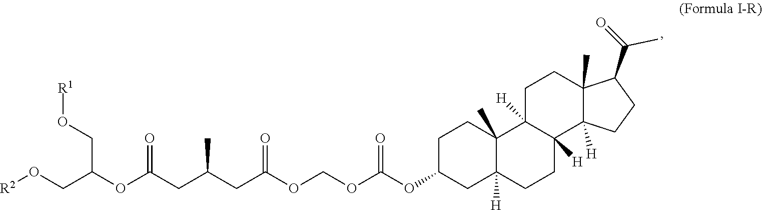

wherein R¹ and R² are as defined above.

In some embodiments, the methods and formulations of Formula I disclosed herein may comprise Formula I-S:

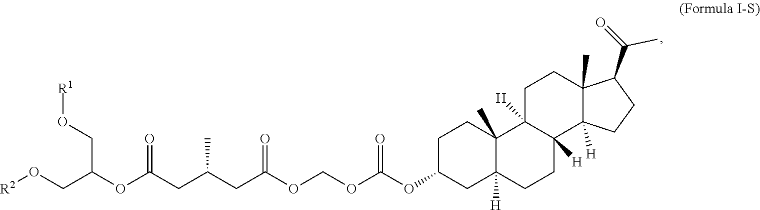

wherein R¹ and R² are as defined above.

In some embodiments the methods and formulations of Formula I disclosed herein comprise a diastereomeric mixture of Formula I-R and Formula I-S. In some embodiments, the diastereomeric mixture of Formula I-R and Formula I-S is at about a 1:1 molar ratio.

### Methods of Treatment

Compound 1 exhibits high oral bioavailability, and as a result exhibits unique pharmacokinetic (PK) and pharmacodynamic (PD) properties as compared to IV-administered allopregnanolone. Without being bound by any particular theory, it is understood that allopregnanolone has anxiolytic effects in mammals when provided exogenously.

Accordingly, in one aspect the methods disclosed herein comprise orally administering to a human subject in need thereof a dose of Compound 1. In some embodiments, the dose of Compound 1 is between 70-500 mg. In some embodiments, the dose of Compound 1 is 70 mg, 125 mg, 140 mg, 250 mg, 280 mg, 375 mg, 420 mg, 500 mg, or in a range between and including any two of these values. In some embodiments, the dose of Compound 1 comprises Compound 1-R. In some embodiments, the dose of Compound 1 comprises Compound 1-S. In some embodiments, the dose of Compound 1 comprises a diasteromeric mixture of Compound 1-R and Compound 1-S.

In another aspect, disclosed herein are methods comprising orally administering to a human subject in need thereof a dose of a compound of Formula I. Amounts of allopregnanolone can be determined based on the total amount of allopregnanolone present in a dose or formulation after accounting for the molecular weight of the prodrug. For example, Compound 1:

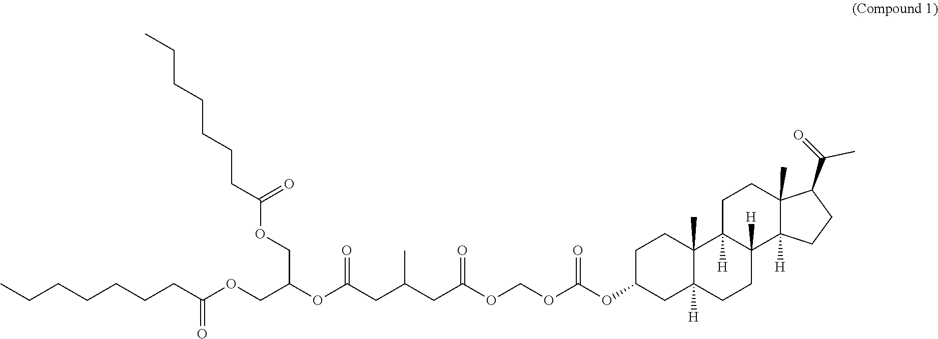

has a molecular weight of 846.55 g/mol. Allopregnanolone has a molecular weight of 317.25 g/mol. Accordingly, 70 mg of Compound 1 is approximately 26.3 mg allopregnanolone and 500 mg of Compound 1 is approximately 187.5 mg allopregnanolone.

Thus, in some embodiments, the dose of a compound of Formula I is in an amount sufficient to deliver between 26.3-187.5 mg of allopregnanolone. In some embodiments, the dose of a compound of Formula I is in an amount sufficient to deliver 26.3 mg, 46.9 mg, 52.5 mg, 93.8 mg, 105.0 mg, 140.6 mg, 157.5 mg, or 187.5 mg of allopregnanolone.

In one aspect, the dose of Compound 1 is administered using a dose titration. For example, a subject may first be administered a low amount of Compound 1. If a dose of Compound 1 is well-tolerated for a period of time, the subject may next be administered a higher amount of Compound 1 (i.e., an up titration). In some embodiments, the period of time is 3-7 days. This up titration can be repeated with a higher dose. If a dose of Compound 1 is not well-tolerated for a period of time, the subject may be administered a lower amount of Compound 1 (i.e., a down titration).

For example, and not by way of limitation, a subject may first be administered 125 mg of Compound 1. If the 125 mg of Compound 1 is well-tolerated for a period of time, the subject may next be administered 250 mg of Compound 1. If the 250 mg of Compound 1 is well-tolerated for a period of time, a subject may next be administered 375 mg of Compound 1. In some embodiments, the period of time is 3-7 days.

Additionally or alternatively, a subject may first be administered 250 mg of Compound 1 once per day. If the 250 mg dose of Compound 1 is well-tolerated for a period of time, the subject may have the dose increased to 375 mg of Compound 1 administered once per day, decreased to 125 mg of Compound 1 administered once per day, or maintained at 250 mg of Compound 1 administered once per day for the duration of treatment. The duration of treatment may be 2, 4, 6, 8, 10, 12 weeks or longer.

Timing of administration is flexible, and Compound 1 may be administered at any point throughout a day. In some embodiments, Compound 1 is administered in the morning. In some embodiments, Compound 1 is administered at night. Compound 1 may be administered with or without food. In preferred embodiments, the dose of Compound 1 is administered at night before bedtime.

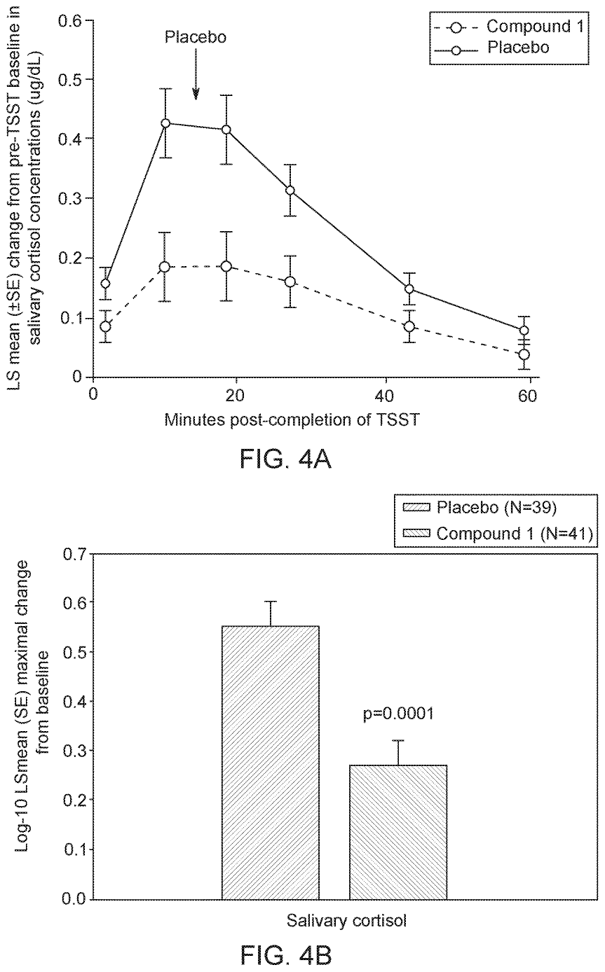

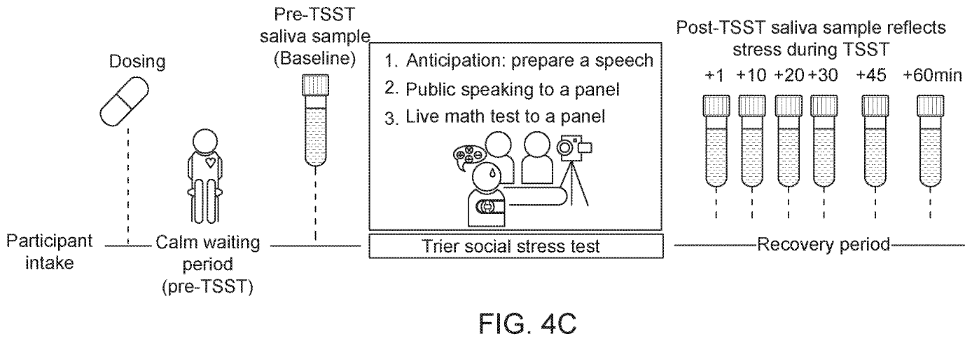

As demonstrated herein, Compound 1 blunts (e.g., reduces/minimizes) an increase in cortisol levels, including for example, cortisol levels increased in response to a stress or stressor. FIG. 4A and FIG. 4B show that administration of Compound 1 to a human subject results in significant blunting of an increase in cortisol levels, levels of which are increased in response to a stressor (for example, in response to a stress test such as the Trier Social Stress Test (TSST) described in Example 2). An increase in cortisol levels after the TSST is a physiological response and an objective biomarker of acute stress.

Disclosed herein is a method of treating a subject, the method comprising administering to the subject in need thereof a dose of Compound 1. In some embodiments, the dose of Compound 1 results in a partial blunting or complete blunting of a stress hormone response (e.g., as measured by an increase in cortisol levels. In some embodiments, the dose of Compound 1 results in a partial blunting of an increase in cortisol levels. In some embodiments, the increase in cortisol levels is in response to a stressor. In some embodiments, the dose of Compound 1 results in a complete blunting of an increase in cortisol levels. In some embodiments, the increase in cortisol levels is in response to a stressor. In some embodiments, the increase in cortisol levels is measured as an increase in salivary cortisol levels. In some embodiments, the increase in salivary cortisol levels is measured using the TSST. In some embodiments, the subject in need thereof has a stress-related disorder. In some embodiments, the stress-related disorder is a specific phobia, social phobia, panic disorder, adjustment disorder, or post-traumatic stress disorder. In some embodiments, the subject in need thereof has an anxiety disorder, including for example separation anxiety disorder, selective mutism, specific phobia, social anxiety disorder, panic disorder, generalized anxiety disorder (GAD), agoraphobia, substance/medication-induced anxiety disorder, anxiety disorder due to another medical condition, other specified anxiety disorder, or unspecified anxiety disorder. In some embodiments, the subject in need thereof has generalized anxiety disorder (GAD). In some embodiments, the subject in need thereof further has a depression or mood disorder or one or more symptoms of a depression or mood disorder. In some embodiments, the depression or mood disorder is selected from major depressive disorder (MDD), bipolar disorder, seasonal affective disorder, cyclothymic disorder, premenstrual dysphoric disorder, persistent depressive disorder, disruptive mood dysregulation disorder, postpartum depression, perimenopausal depression, or a depression related to a medical illness. In some embodiments, the subject in need thereof has generalized anxiety disorder (GAD) which is originated, induced, or exacerbated by stress, a stressor, or a stress-related disorder. In some embodiments, the subject in need thereof has generalized anxiety disorder with a mood or depression disorder or one or more symptoms of a mood or depression disorder. In some embodiments, the depression or mood disorder is major depressive disorder (MDD), bipolar disorder, seasonal affective disorder, cyclothymic disorder, premenstrual dysphoric disorder, persistent depressive disorder, disruptive mood dysregulation disorder, postpartum depression, perimenopausal depression, or a depression related to a medical illness.

It is estimated that approximately 280M people worldwide are affected by major depressive disorder and approximately 301M worldwide people are affected by anxiety disorders (WHO; depressive disorder fact sheet (2023)). Of those, ~120M are estimated to have MDD with anxiety disorders, such as MDD with anxiety (MDD with anxious distress) (Kessler et al., (2015) Science 24(3):210-216). Subjects with depression (i.e., subjects with MDD) have a higher suicide risk compared to that of the general population (20× higher; American Association of Suicidology (2009)). Further, subjects having MDD with anxiety are less likely to achieve remission, are slower to respond to treatment, and poor quality of life as compared to subjects with MDD without anxiety or anxious distress (Hopwood M (2023) Neurol Ther 12(suppl 1):5-12. Accordingly, in some embodiments, the subject in need thereof has generalized anxiety disorder with a mood or depression disorder or one or more symptoms of a mood or depression disorder, such as, but not limited to major depressive disorder. In some embodiments, the subject in need thereof has major depressive disorder with anxiety (MDD with anxious distress).

In some embodiments, the subject in need thereof has generalized anxiety disorder which is originated, induced, or exacerbated by stress or a stress-related disorder with a mood or depression disorder or one or more symptoms of a mood or depression disorder such as, but not limited to, major depressive disorder.

An increase in cortisol levels after a stressor, such as the TSST, is a physiological response and an objective biomarker of acute stress. The oral administration of Compound 1 achieves a statistically significant reduction in the stress hormone response as measured by salivary cortisol levels. This reduction in the stress hormone response indicates its potential as a treatment for a range of anxiety disorders. In one aspect, disclosed herein is a method of treating an anxiety disorder comprising orally administering to a human subject in need thereof a dose of Compound 1. In some embodiments, the dose of Compound 1 is between 70-500 mg. In some embodiments, the anxiety disorder is generalized anxiety disorder (GAD). In some embodiments, the human subject may further have a depression or mood disorder or one or more symptoms of a depression or mood disorder. In some embodiments, the depression or mood disorder is selected from major depressive disorder (MDD), bipolar disorder, seasonal affective disorder, cyclothymic disorder, premenstrual dysphoric disorder, persistent depressive disorder, disruptive mood dysregulation disorder, postpartum depression, perimenopausal depression, or a depression related to a medical illness. In some embodiments, the depression or mood disorder is MDD. In some embodiments, the depression or mood disorder is MDD with anxiety (MDD with anxious distress).

In one aspect, disclosed herein is a method of treating an anxiety disorder, comprising orally administering to a human subject in need thereof a dose of Compound 1, wherein the subject has generalized anxiety disorder (GAD) with one or more depression or mood disorders or one or more symptoms of a depression or mood disorder. In some embodiments, the depression or mood disorder is MDD. In some embodiments, the depression or mood disorder is MDD with anxiety (MDD with anxious distress).

In one aspect, disclosed herein is a method of reducing the stress hormone response in a human subject comprising orally administering to the human subject in need thereof a dose of Compound 1. In some embodiments, the reduction of the stress hormone response is measured as a reduction in cortisol, e.g., salivary cortisol. In some embodiments, the dose of Compound 1 is between 70-500 mg. In some embodiments, the dose of Compound 1 is 125 mg. In some embodiments, the dose of Compound 1 is 250 mg. In some embodiments, the dose of Compound 1 is 375 mg. In some embodiments, the human subject has a stress-related disorder or an anxiety disorder. In some embodiments, the human subject has GAD. In some embodiments, the human subject has GAD with one or more depression or mood disorders or one or more symptoms of a depression or mood disorder. In some embodiments, the depression or mood disorder is MDD. In some embodiments, the depression or mood disorder is MDD with anxiety (MDD with anxious distress).

Although cortisol blunting following a TSST has been observed with certain benzodiazepines (alprazolam, see Fries et al., 2006), it is not a universally observed effect of GABA_{A} agonists or GABA_{A} PAMs. For example, Etifoxine is a non-benzodiazepine GABA_{A} PAM that does not blunt an increase in salivary cortisol levels following a TSST.

Furthermore, current standard-of-care treatments for anxiety have shown serious drawbacks, including inconsistent efficacy, tolerance issues, poor tolerability (adverse events) and potential for abuse. Given its unique pharmacokinetic (PK) and pharmacodynamic (PD) properties (e.g., including its high oral bioavailability, amongst others), the administration of Compound 1 provides several advantages as compared to benzodiazepines or other therapeutics used in the treatment of stress, anxiety, or stress-related disorders (e.g., β-blockers like propranolol). For example, the extent of cortisol blunting (i.e., the extent to which increases in salivary cortisol levels are blunted in response to a stressor) by an agent may vary, and can include partial blunting or complete blunting (e.g., as afforded by alprazolam). Compound 1 has shown the ability to partially blunt increases in cortisol levels. In certain instances, partial blunting in response to a stressor may be advantageous. For example, and without wishing to be bound by any particular theory, Compound 1, due to its partial blunting effect, may be less addictive than benzodiazepines which result in complete blunting of a stress hormone response, e.g., cortisol level increases in response to stress. This may allow for chronic treatment with less potential for abuse. Also, Compound 1 has a Tmax of ~3-5 hours (see Example 1), which provides a delayed onset of therapeutic effect and allows for a more flexible dosing regimen (i.e., affords the potential for a longer time interval between doses). As described in Example 2 and FIG. 4C, patients received a dose of Compound 1 approximately 3-4 hours prior to experiencing a stressor (e.g., a TSST). In contrast, benzodiazepines and β-blockers require administration 30 minutes to 1 hour prior to undergoing a stressor (e.g., a TSST). Compound 1 further allows for blunting of cortisol levels with reduced euphoria, reduced loss of consciousness, reduced somnolence, reduced dizziness, and/or reduced balance disorder (e.g., reduced incidence of nervous system disorder-related AEs).

### Formulation

While Compound 1 is designed for oral administration, it is understood that Compound 1 may be prepared as a composition with one or more pharmaceutically acceptable carriers, adjuvants, or vehicles as are known in the art. To aid in delivery, Compound 1 may be formulated in a lipid-based formulation.

Lipid formulations may contain lipids and/or surfactants, optionally with co-solvents, and are generally categorized into 4 types. Type I formulations include lipids which require digestion, such as mono-, di-, and triglycerides and combinations thereof. Type II formulations are water-insoluble self-emulsifying drug delivery systems (SEDDS) which contain lipids in addition to water insoluble surfactants. Type III formulations are SEDDS or self-microemulsifying drug delivery systems (SMEDDS) which contain lipids in addition to water-soluble surfactants and/or co-solvents. Type IV formulations contain predominantly hydrophilic surfactants and co-solvents such as PEG and propylene glycol. Examples of lipids suitable for use with Compound 1 are further described in WO2021/159021.

The oral dosage form of Compound 1 can be administered in any orally acceptable dosage form, including but not limited to capsules, tablets, suspensions or solutions as are known in the art.

In one embodiment, Compound 1 is prepared in formulation with Peceol™ (glycerol monooleate, available from Gattefosse), Kolliphor® RH40 (a nonionic solubilizer obtained by the reaction of 1 mole of hydrogenated castor oil with 40 moles of ethylene oxide, also referred to as polyoxyl 40 hydrogenated castor oil or macrogolglycerol hydroxystearate and available from BASF Corp.), and super-refined sesame oil.

In one embodiment, each gram of formulation contains 300 mg of Compound 1, 189 mg of Peceol™, 336 mg of Kolliphor® RH40, and 175 mg of super-refined sesame oil. In some embodiments, the formulation is filled into capsules. In one embodiment, the capsule is a soft-gelatin capsule. By way of illustration, in some embodiments, the capsule comprises a formulation comprising 125 mg, 250 mg, or 375 mg of Compound 1.

### EXAMPLES

The following examples demonstrate the high oral bioavailability of Compound 1 and its efficacy in treating the disorders disclosed herein.

### Example 1: A Study to Assess the Safety, Tolerability, and PK/PD Profile of Compound 1 in Humans

A three-part study to assess the safety, tolerability, and PK/PD properties of Compound 1 in humans was performed. Part 1 was a randomized, double-blinded, placebo-controlled, single ascending dose (SAD) phase. Part 2 was a randomized, open-label phase to assess the effect of food on Compound 1. Part 3 was a randomized, double-blinded, placebo-controlled multiple-ascending dose phase.

Compound 1 was generally well tolerated across dose levels tested with no deaths, no severe or serious drug-related adverse events, no discontinuations due to treatment-emergent adverse events (TEAEs), and no hepatic, cardiac, or renal-related treatment-emergent adverse events.

### Part 1: Single Ascending Dose (SAD)

The SAD phase utilized a crossover, dose-escalation design with 3 periods. Dosing was arranged in 1 of 3 sequences such that each subject would receive 1 administration of placebo and 2 administrations of Compound 1 (randomized as a low, medium, or high dose, per cohort). A washout period of at least 7 days was included between each administration. Compound 1 or placebo was administered orally following an overnight fast of at least 10 hours. Cohort 2 and 2b were dosed following a standard meal (i.e., a fed state). The cohort design and dosing scheme is described in Table 1.

TABLE 1
SAD Cohort and Dosing Scheme

| | Number of | Treatment | Treatment Period | | |
|---|---|---|---|---|---|
| Cohort | Subjects | Sequence | Period 1 | Period 2 | Period 3 |
| 1 | 9 | 1 | Placebo | 140 mg | 280 mg |
| (Fasted) | | 2 | 70 mg | Placebo | 280 mg |
| | | 3 | 70 mg | 140 mg | Placebo |
| 2 | 9 | 1 | Placebo | 420 mg | 560 mg |
| (Fed) | | 2 | 280 mg | Placebo | 560 mg |
| | | 3 | 280 mg | 420 mg | Placebo |
| 2b | 9 | 1 | Placebo | 1000 mg | N/A* |
| (Fed) | | 2 | 750 mg | Placebo | N/A* |
| | | 3 | 750 mg | 1000 mg | N/A* |

*Period 3 was not performed for cohort 2b as initial PK data indicated the 1000 mg dose was greater than dose-proportional as compared to the 750 mg dose.

Part 1 Primary Objectives: to evaluate the safety and tolerability of single oral doses of Compound 1 and to determine the maximum tolerated dose (MTD) following ascending single oral doses of Compound 1. Part 1 Primary Endpoints: incidence, severity, and duration of treatment-emergent adverse events (TEAEs), serious adverse events (SAEs), and incidence of clinically significant vital signs, pulse oximetry, ECGs, safety laboratory, or physical examination findings and MTD of Compound 1 in healthy subjects.

Part 1 Secondary Objectives: to characterize the pharmacokinetic (PK) profile Compound 1 following single oral dosing. Part 1 Secondary Endpoints: PK parameter determination for Compound 1, combined allopregnanolone-containing molecules, and allopregnanolone, and include, but are not limited to: maximum plasma concentration (Cmax); time to maximum concentration (Tmax); terminal elimination half-life (t_{1/2}); area under the plasma concentration-time curve (AUC) from time zero (from the time of dosing) to the last measurable concentration (AUC0-last); AUC from time zero to infinity (AUC_{0-inf}); estimation of the elimination rate constant (λz); and apparent total clearance (CL/F); the apparent volume of distribution (Vz/F).

Part 1 Exploratory Objectives: To characterize the pharmacodynamic (PD) effects, and the PK/PD relationship, of Compound 1 on select clinical outcome assessments (COAs) of central nervous system effects following ascending single oral doses of Compound 1 (e.g., Stanford Sleepiness Scale (SSS), Modified Observer's Assessment of Alertness/Sedation Scale (MOAA/S), Sleep Quality Scale (SQS)). Part 1 Exploratory Endpoints: Changes from Baseline in SSS, MOAA/S, and/or SQS.

### Part 1 PK Summary

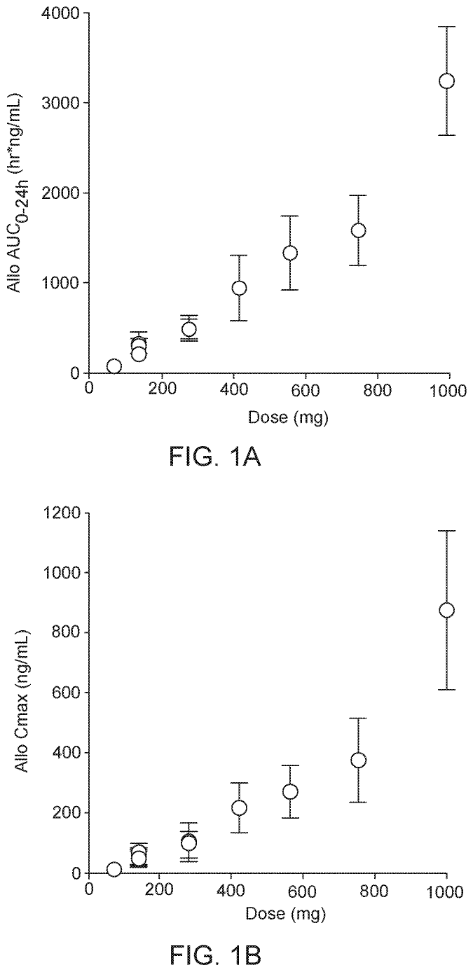

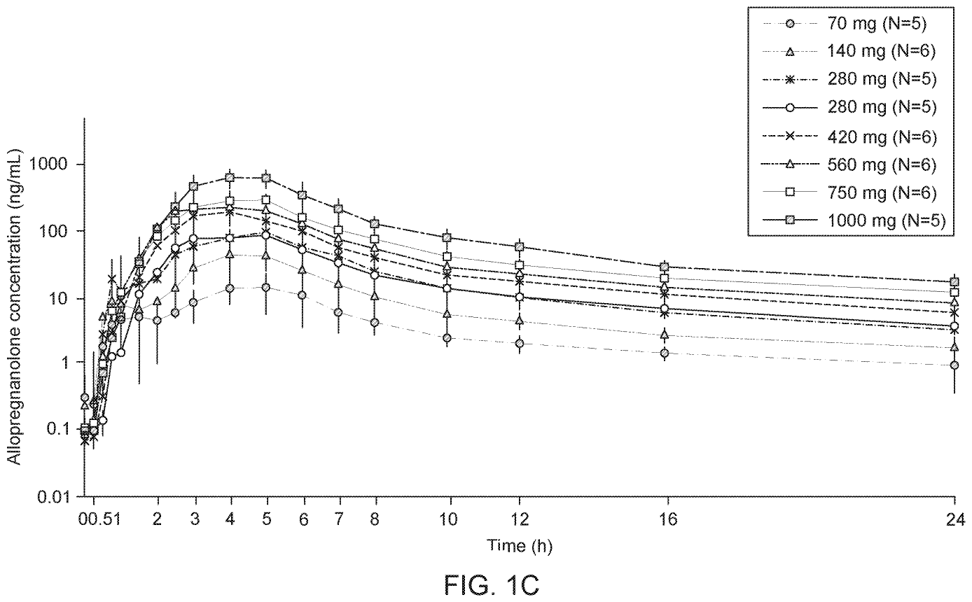

Mean plasma allopregnanolone (free allopregnanolone) and combined allopregnanolone (allopregnanolone liberated from all allopregnanolone-containing molecules during analysis) concentrations increased at a dose proportional rate up to 560 mg; in the range of 750 to 1000 mg there was a greater than dose-proportional increase observed in several PK parameters. FIG. 1A-1B demonstrate the observed dose proportionality for both AUC (FIG. 1A) and Cmax (FIG. 1B). Allopregnanolone and combined allopregnanolone rapidly appeared in the blood and reached peak mean concentrations between 3-5 hours post-dose, followed by exponential decline. The concentration of allopregnanolone over time for the doses tested in Part 1 is shown in FIG. 1C. The shape of the concentration over time profile of Compound 1 was approximately similar for all doses. The plasma concentrations of combined allopregnanolone were markedly higher than those for allopregnanolone.

Maximal concentrations and exposure values were significantly larger for combined allopregnanolone as compared to allopregnanolone (at least 10 times higher). The median Tmax and mean t_{1/2} for both analytes were not impacted by dose level and did not exhibit any dose-specific trends. Allopregnanolone and combined allopregnanolone AUC₀₋ₜ, AUC_{0-inf}, and Cmax each increased as the dose level increased. When calculated against dose level (AUC_{0-inf}/Dose and Cmax/Dose) it was determined that there was an increase in dose corrected parameters over the dose range. Overall, for each analyte and the PK parameters tested (Cmax, AUC₀₋ₜ, and AUC_{0-inf}), the increases were slightly greater than dose-proportional, particularly in the dose range of 750-1000 mg of Compound 1. A summary of allopregnanolone PK parameters for Part 1 is provided in Table 2.

TABLE 2
PK Parameters for Single Ascending Dose Study

Cohort 1

| | Period 1 | Period 2 | Period 3 |
|---|---|---|---|
| PK Parameter | (70 mg) | (140 mg) | (280 mg) |
| Mean Cₘₐₓ (ng/mL) | 17.7 | 49.3 | 111 |
| Median Tₘₐₓ (hr) | 4 | 5 | 4 |
| Mean Half-life (hr) | 35.2 | 37.6 | 25.1 |
| Mean AUC₀₋₂₄ | 91.3 | 239 | 506 |
| (hr*ng/mL) | | | |
| Mean AUC_{inf} | 150 | 352 | 625 |
| (hr*ng/mL) | | | |

Cohort 2

| | Period 1 | Period 2 | Period 3 |
|---|---|---|---|
| PK Parameter | (280 mg) | (420 mg) | (560 mg) |
| Mean Cₘₐₓ (ng/mL) | 103 | 219 | 273 |
| Median Tₘₐₓ (hr) | 3 | 4 | 3.5 |
| Mean Half-life (hr) | 22.8 | 25.3 | 26.2 |
| Mean AUC₀₋₂₄ | 497 | 953 | 1340 |
| (hr*ng/mL) | | | |
| Mean AUC_{inf} | 633 | 1170 | 1640 |
| (hr*ng/mL) | | | |

Cohort 2b

| | Period 1 | Period 2 |
|---|---|---|
| PK Parameter | (750 mg) | (1000 mg) |
| Mean Cₘₐₓ (ng/mL) | 378 | 878 |
| Median Tₘₐₓ (hr) | 4 | 4 |
| Mean Half-life (hr) | 22 | 21.4 |
| Mean AUC₀₋₂₄ | 1590 | 3250 |
| (hr*ng/mL) | | |
| Mean AUC_{inf} | 2000 | 3790 |
| (hr*ng/mL) | | |

### Part 2: Food Effect

The effect of food (both low-fat and high-fat meals) on the safety, tolerability, and PK of Compound 1 was assessed using a 3 period crossover design. Dosing was arranged in 1 of 3 sequences such that each subject would receive 3 administrations of Compound 1 with a minimum 7 day washout period between doses. The cohort design and dosing scheme for Part 2 is shown in Table 3.

TABLE 3
Food Effect Cohort and Dosing Scheme

| | Number of | Treatment Period | | |
|---|---|---|---|---|
| Cohort | Subjects | Period 1 | Period 2 | Period 3 |
| 3 | 3 | 140 mg Fed (HF) | 140 mg Fed (LF) | 140 mg Fasted |
| | 3 | 140 mg Fed (LF) | 140 mg Fasted | 140 mg Fed (HF) |
| | 3 | 140 mg Fasted | 140 mg Fed (HF) | 140 mg (LF) |

Part 2 Primary Objective: To estimate the effect of a high-fat (HF) and low-fat (LF) meal on the bioavailability of a single oral dose of Compound 1.

Part 2 Secondary Objective: To assess the safety, tolerability, and PK of a single dose of Compound 1 under fed (HF), fed (LF), and fasted conditions.

Part 2 Exploratory Objectives: To characterize the pharmacodynamic effects of Compound 1 on select clinical outcome assessments of central nervous system effects following a single dose of Compound 1 (e.g. SSS, MOAA/S, C-SSRS, SQS) under fed HF, fed LF, and fasted conditions.

The fed HF meal consisted of approximately 800-1000 calories, consisting of a macronutrient content of approximately 150, 250, and 500-600 calories each from protein, carbohydrates, and fat, respectively. The fed LF meal consisted of approximately 400 500 calories, consisting of approximately 25% calories from fat (approximately 11-14 g fat), and a variable caloric content derived from protein and carbohydrates such that the total calories does not exceed the range of 400-500 calories.

### Part 2 PK Analysis Summary

Mean plasma allopregnanolone and combined allopregnanolone concentrations increased slightly in fed versus fasted states. Concentrations of allopregnanolone and combined allopregnanolone rapidly appeared in the blood and reached peak concentrations between 3-5 hours post-dose and were not significantly impacted by the fed state. Plasma concentrations of combined allopregnanolone were markedly higher than those for allopregnanolone.

Maximal concentrations and exposure values were significantly larger for combined allopregnanolone as compared to allopregnanolone (approximately 15 times higher). The food effect was minimal: Allopregnanolone and combined allopregnanolone AUC₀₋ₜ and AUC_{0-inf} and Cmax each slightly increased in the fed (LF) group compared to the fasted and fed (HF) groups by ~1.3 to 1.5-fold. A summary of allopregnanolone PK parameters for Part 2 is provided in Table 4.

TABLE 4
PK Parameters for Food Effect Study

Cohort 3 (140 mg)

| PK Parameter | Fasted | Fed (HF) | Fed (LF) |
|---|---|---|---|
| Mean Cₘₐₓ (ng/mL) | 56.9 | 55.2 | 74.7 |
| Median Tₘₐₓ (hr) | 5 | 4 | 3 |
| Mean Half-life (hr) | 31.9 | 26.1 | 26.4 |
| Mean AUC₀₋₂₄ | 224 | 308 | 341 |
| (hr*ng/mL) | | | |
| Mean AUC_{inf} | 288 | 410 | 434 |
| (hr*ng/mL) | | | |

### Part 3: Multiple Ascending Dose (MAD)

The safety, tolerability, and PK of Compound 1 administered as multiple doses (up to 7 days) was determined. Subjects in cohorts 4-6 were administered Compound 1 once daily in the morning (QAM) in a fed state. Subjects in cohorts 7-8 were administered Compound 1 once daily in the evening (QHS) in a fed state. The cohort design and dosing scheme for Part 3 is shown in Table 5.

TABLE 5
MAD Cohort and Dosing Scheme

| | |
|---|---|
| Compound | Number of Subjects |

| Cohort | 1 Dose | Compound 1 | Placebo |
|---|---|---|---|
| 4 | 250 mg QAM | 9 | 3 |
| 5 | 375 mg QAM | 9 | 3 |
| 6 | 500 mg QAM | 9 | 3 |
| 7 | 375 mg QHS | 9 | 3 |
| 8 | 375 mg QHS | 9 | 3 |

Part 3 Primary Objective: to evaluate the safety and tolerability of multiple oral doses of Compound 1 and to determine the maximum tolerated dose of Compound 1 following multiple oral doses. Part 3 Primary Endpoints: incidence, severity, and duration of treatment-emergent adverse events, significant adverse events, and incidence of clinically significant vital signs, pulse oximetry, ECGs, safety laboratory, or physical examination findings, and the maximum tolerated dose of Compound 1 in healthy subjects.

Part 3 Secondary Objective: to characterize the PK profile of Compound 1 following multiple oral dosing. Part 3 Secondary Endpoints: PK parameters for Compound 1, combined allopregnanolone, and allopregnanolone were determined, including but not limited to observed maximum plasma concentration in the dosing interval at steady state; observed minimum concentration in the dosing interval at steady state; average steady state concentration; the quantifiable concentration at the end of the dosing interval at steady state; the time to reach maximum concentration in the dosing interval at steady state; area under the drug concentration-time curve from time zero to the last measurable concentration at steady state; area under the plasma concentration-time curve over a dosing interval at steady state; AUC_{0-inf}, the percentage of the AUC that has been extrapolated beyond the last observed data point; λz; t_{1/2}; apparent total plasma clearance at steady state, apparent terminal volume of distribution at steady state, mean residence time (MRT), swing, and fluctuation %.

Part 3 Exploratory Objective: to characterize the PD effects, and the PK/PD relationships of Compound 1 on select effects following multiple oral doses of Compound 1. Part 3 Exploratory Endpoints: changes from baseline in qEEG, SSS, MOAA/S, the SQS, Cogstate, Body Sway, and video-oculography (VOG) assessment.

### Part 3 PK Analysis Summary

The mean concentration of allopregnanolone and combined allopregnanolone rapidly appeared in the blood and reached peak mean concentrations between 3-5 hours post-dose. Mean allopregnanolone and combined allopregnanolone concentration values increased with increasing Compound 1 dose. Mean plasma peak concentration values for allopregnanolone were higher on Day 1 compared to those on Day 7 across cohorts 4-6. In contrast, mean plasma peak concentration values for combined allopregnanolone exhibited higher levels on Day 7 compared to Day 1, suggesting a small degree of accumulation. No apparent differences were observed between QAM and QHS dosing.

The maximal concentrations and exposure values were significantly larger for combined allopregnanolone compared to allopregnanolone. Allopregnanolone and combined allopregnanolone AUC₀₋ₜ, AUC₀₋₂₄, and AUC_{0-τ} and Cmax,ss each increased with increasing dose of Compound 1 over 7 days. The increases in systemic exposure were consistent with the findings in Part 1.

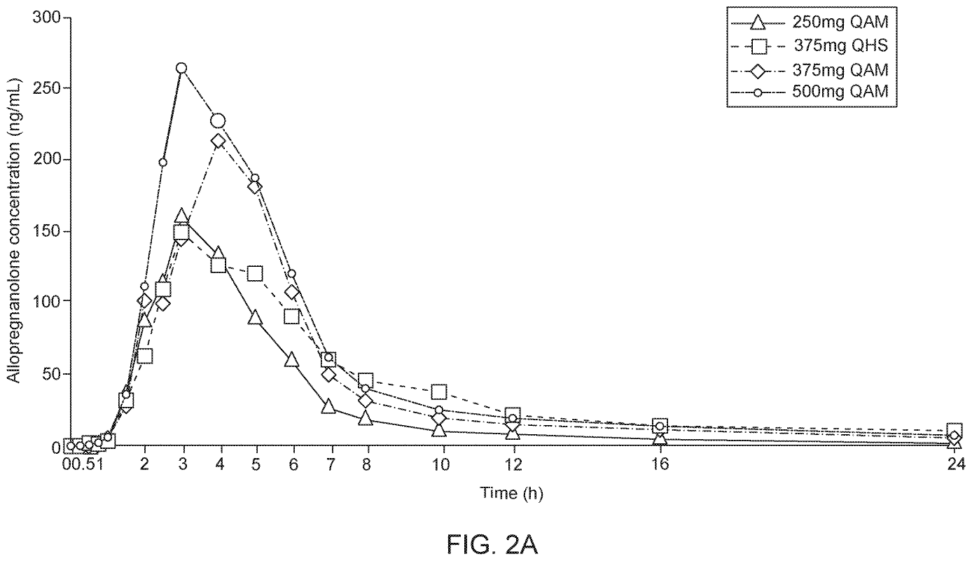

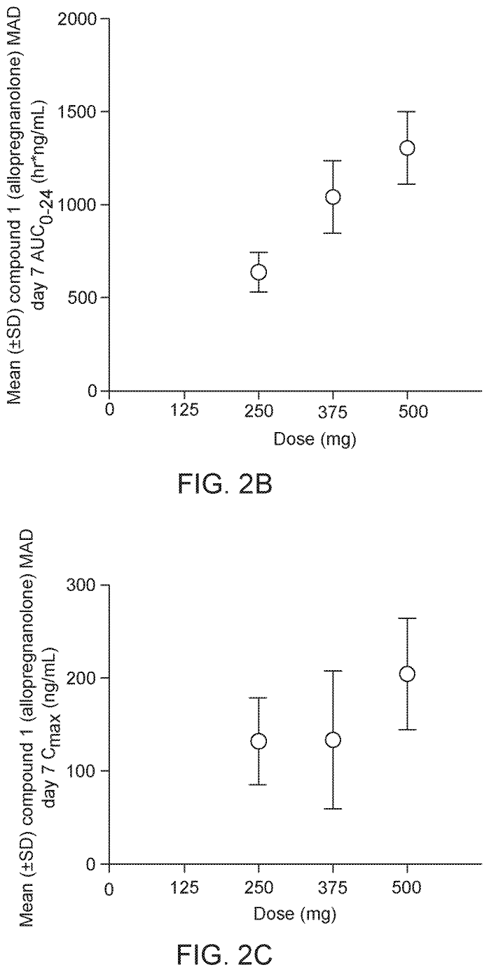

Both allopregnanolone and combined allopregnanolone exhibited minimal accumulation upon multiple dosing, reaching steady state by Day 2 and Day 3 following once daily administration. Allopregnanolone plasma concentrations over time are shown in FIG. 2A. The mean AUC and Cmax at day 7 across the doses tested are shown in FIG. 2B and FIG. 2C, respectively. A summary of allopregnanolone PK parameters for Part 3 is provided in Table 6.

TABLE 6
PK Parameters for Multiple Ascending Dose Study

| | | |
|---|---|---|
| PK Parameter | Day 1 | Day 7 |

Cohort 4 (250 mg QAM)

| | | |
|---|---|---|
| Mean Cₘₐₓ (ng/mL) | 199 | 132 |
| Median Tₘₐₓ (hr) | 3 | 4 |
| Mean Half-life (hr) | — | 23.9 |
| Mean AUC₀₋₂₄ (hr*ng/mL) | 698 | 699 |

Cohort 5 (375 mg QAM)

| | | |
|---|---|---|
| Mean Cₘₐₓ (ng/mL) | 283 | 171 |
| Median Tₘₐₓ (hr) | 4 | 4 |
| Mean Half-life (hr) | — | 23.6 |
| Mean AUC₀₋₂₄ (hr*ng/mL) | 1010 | 992 |

Cohort 6 (500 mg QAM)

| | | |
|---|---|---|
| Mean Cₘₐₓ (ng/mL) | 290 | 205 |
| Median Tₘₐₓ (hr) | 3 | 3.5 |
| Mean Half-life (hr) | — | 28.7 |
| Mean AUC₀₋₂₄ (hr*ng/mL) | 1260 | 1240 |

Cohort 8 (375 mg QHS)

| | | |
|---|---|---|
| Mean Cₘₐₓ (ng/mL) | 174 | 180 |
| Median Tₘₐₓ (hr) | 3 | 3 |
| Mean Half-life (hr) | — | 26.1 |
| Mean AUC₀₋₂₄ (hr*ng/mL) | 845 | 1110 |

Participants were assessed using metrics as outlined below. The SSS is a one item self-report questionnaire that measures and quantifies progressive steps in the level of sleepiness throughout the day. Subjects select one of seven statements that best represents their level of perceived sleepiness at a given timepoint, as depicted in Table 7.

TABLE 7
Stanford Sleepiness Scale Ratings

| Degree of Sleepiness | Scale Rating |
|---|---|
| Feeling active, vital, alert, | 1 |
| or wide awake | |
| Functioning at high levels, but | 2 |
| not at peak; able to concentrate | |
| Awake, but relaxed; responsive | 3 |
| but not fully alert | |
| Somewhat foggy, let down | 4 |
| Foggy; losing interest in remaining | 5 |
| wake; slowed down | |
| Sleepy, woozy, fighting sleep; | 6 |
| prefer to lie down | |
| No longer fighting sleep, sleep | 7 |
| onset soon; having dream-like thoughts | |
| Asleep | X |

The MOAA/S is a clinical outcome assessment that measures a subject's level of alertness/sedation as determined by a rater; in its modified form, it uses only the responsiveness component of the original Observer's Assessment of Alertness/Sedation Scale. Responsiveness, as a measure of sedation, was assessed and scored on a scale of 0 to 5 as outlined in Table 8.

TABLE 8
MOAA/S Score Descriptions

| Description | Score |
|---|---|
| Responds readily to name | 5 |
| spoken in normal tone | |
| Lethargic response to name | 4 |
| spoken in normal tone | |
| Responds only after name is | 3 |
| called loudly and/or repeatedly | |
| Responds only after mild | 2 |
| prodding or shaking | |
| Responds only after painful | 1 |
| trapezius squeeze | |
| No response after painful | 0 |
| trapezius squeeze | |

The SQS is a single-item scale which has been demonstrated to possess favorable measurement characteristics to assess sleep quality. It measures clinically meaningful changes in sleep quality without significantly increasing the burden of clinical trial participants. The scale was administered in the morning, and subjects are asked to rate their sleep quality of the past 7 days, ranging from terrible (score of 0), poor (scores of 1-3), fair (scores of 4-6), good (scores of 7-9), and excellent (score of 10).

The Columbia-Suicide Severity Rating Scale (C-SSRS) is a validated tool designed to quantify the severity of suicidal ideation and behavior. This scale was administered by individuals who have received training in its administration, as described in further detail at https://cssrs.columbia.edu/the-columbia-scale-c-ssrs/about-the-scale/.

Quantitative Electroencephalogram (qEEG or EEG) has gained prominence as a useful biomarker in human drug studies as it is continuous, objective, repeatable, sensitive, reproducible, non-invasive and highly translatable from nonclinical data. EEG serves as biomarker in this protocol and provides PD outcomes for PK-PD modeling, thus enabling a more thorough understanding of the pharmacological effects of Compound 1. In addition, EEG can provide useful safety information regarding detection of epileptiform activity and any pro-convulsive effects of the study drug. Data outputs included spectral analysis and flat brain mapping, among others.

EEG was collected at multiple time points before and after dosing on Day 1 and Day 7 of the MAD portion of the study except for Cohort 8. For a given recording, the subject was fitted with a cap containing the electrodes. For each session, recordings were collected for approximately 15 minutes while subjects are sitting quietly, and include portions with eyes open and eyes closed.

The Cogstate™ neuropsychiatric assessment battery that was designed for this protocol consists of 5 psychomotor tests that identify potential pharmacologically-induced CNS depressant effects. The data constitute a biomarker of such CNS depression and results may be correlated with other biomarker measures such as qEEG. The following provides details on the nature and operation of each test. These were performed for the MAD study cohorts except for Cohort 8. For each one, the participant was encouraged to work as quickly as they can and be as accurate as possible. The software measures the speed and accuracy of each response.

The Detection test is a measure of psychomotor function and uses a well-validated simple reaction time paradigm (processing speed) with playing card stimuli. In this test, the playing cards all depict the same joker. The subject is asked to press the Yes key as soon as the card in the center of the screen turns face up. The test is typically completed in 3 minutes.

The Identification test is a measure of visual attention and uses a well-validated choice reaction time paradigm with playing card stimuli. In this test, the playing cards are all either red or black jokers. The subject is asked whether the card displayed in the center of the screen is red. The subject responds by pressing the Yes key when the joker card is red and No when it is black. The test is typically completed in 3 minutes.

The One Card Learning test is a measure of visual learning and uses a well-validated pattern separation paradigm with playing card stimuli. In this test, the playing cards are identical to those found in a standard deck of 52 playing cards (without the joker cards). The subject is asked whether the card displayed in the center of the screen was seen previously in this test. The subject responds by pressing the Yes or No key. The test is typically completed in 6 minutes.

The One Back test is a measure of working memory and uses a well-validated n-back paradigm with playing card stimuli. The subject is asked whether the next card displayed in the center of the screen is the same as the card presented immediately previously. The subject responds by pressing the Yes or No key. The test typically requires 4 minutes to complete.

The Groton Maze Learning Test measures executive function and problem solving using a maze learning paradigm. A 10×10 grid of tiles is presented to the participant on the screen. A 28-step pathway is hidden among these tiles. The participant must move one step at a time from the start toward the end by touching a tile next to their current location. If the correct move corresponding to the hidden path is made, a green checkmark appears and if the move is incorrect, a red cross is revealed. Once completed, they are returned to the start location to repeat the test and must try to remember the pathway they have just completed. The software records each move as an error or as a correct move. The duration of the test is typically 7 minutes.

Body Sway is assessed as a measure of a subject's slight postural movements in order to retain balance either statically in a neutral position or dynamically while in motion. Body Sway is typically measured by total displacement of the center of body mass relative to the base of support over time. Posturography is an objective, sensitive, reliable, and non-invasive method designed to assess the effects of drugs and alcohol on body sway and vigilance. Body sway is recorded using a computerized force-platform. Using specific positioners, bare feet subjects are asked to stand still and motionless, looking at a cue placed in front of them. Measurement of body sway (one minute with eyes open and one minute with eyes closed) is recorded as recommended by the International Society of Postuography. The length and area of the postural oscillations are then calculated.

Video-oculography (VOG) is a non-invasive, visual method of measuring horizontal, vertical, and torsional components involved in eye tracking, making use of a head-mounted mask equipped with cameras. VOG was used to measure 3 assessment metrics: horizontal saccades (movement of eyes towards a point of fixation), horizontal anti-saccades (movement of eyes away from a point of fixation), and horizontal and vertical pursuits (fixation of eyes on a moving object).

Binocular (both eyes) movements viewing were measured during the saccades test. During this 1-min trial, the visual target (small square) will jump from 0° to 10° to the right or to the left in a pseudorandom sequence. Duration of the target on screen vary pseudo-randomly from 1,500 to 2,000 ms to minimize anticipation of the subject. Then the visual target will be extinguished during 200 ms before the next move starting from the center. The participant is instructed to track the target on screen from center to side and from side to center without anticipation. The antisaccades test is identical to the saccades test in its procedure (with 10° jumps of the target) except that there is no extinction of the target. The participant is instructed to look at the opposite side of the target, approximately at equal distance from the center and must resist looking at the target.

Binocular movements viewing were measured during the pursuit tests. During these tests, a target is continuously moving on screen on the horizontal (horizontal pursuit test) or vertical axis (vertical pursuit test). Each test lasts 10 seconds.

### Pharmacodynamic Results (Parts 1-3)

Cohorts 1, 2, and 2b (Part 1) exhibited a dose-dependent increase from baseline in the Stanford Sleepiness Scale (SSS) and a decrease from baseline in the MOAA/S score. The highest Compound 1 doses had the largest magnitude of increase or decrease, respectively. This indicates greater sleepiness and less alertness/agitation in subjects receiving the higher doses of Compound 1. Maximum changes were observed around 3-5 hours post-dose, which correlates with the time of peak exposure based on corresponding PK results. All treatment groups returned to baseline levels by 12 hours post-dose.

There was no apparent difference in mean change from baseline between the fed (HF or LF) and fasted states (cohort 3).

Cohorts 4, 5, 7, and 8 (Part 3) receiving 250 mg and 375 mg doses exhibited a low magnitude of change from baseline for both SSS and MOAA/S during the early days of dosing, with no meaningful differences between the two dose levels with respect to changes in the SSS and MOAA/S over time. Cohort 6 (Part 3) receiving 500 mg doses demonstrated a greater magnitude of effect from baseline for the SSS and MOAA/S metrics as compared to the lower doses. Notably, the effects on SSS and MOAA/S decreased in all treatment groups over time. Placebo groups demonstrated little change from baseline across treatment time.

There were no consistent trends between Compound 1 and sleep quality as measured by SQS, with no meaningful improvements or worsening observed in any cohorts.

No subjects across all parts (1-3) reported any suicidal ideation or suicidal behavior.

Slight effects on body sway were observed on day 1 but were absent on day 7.

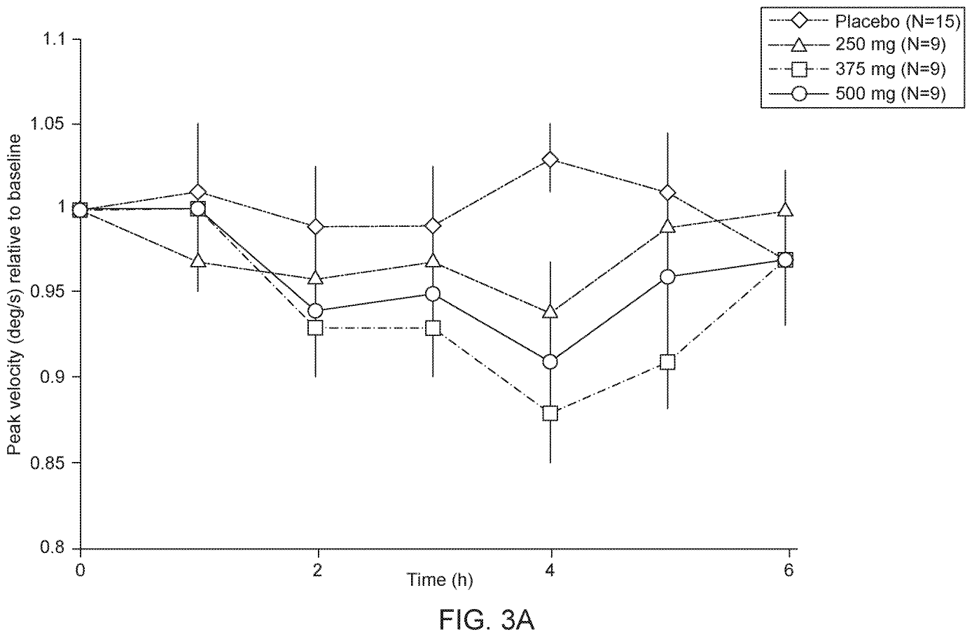

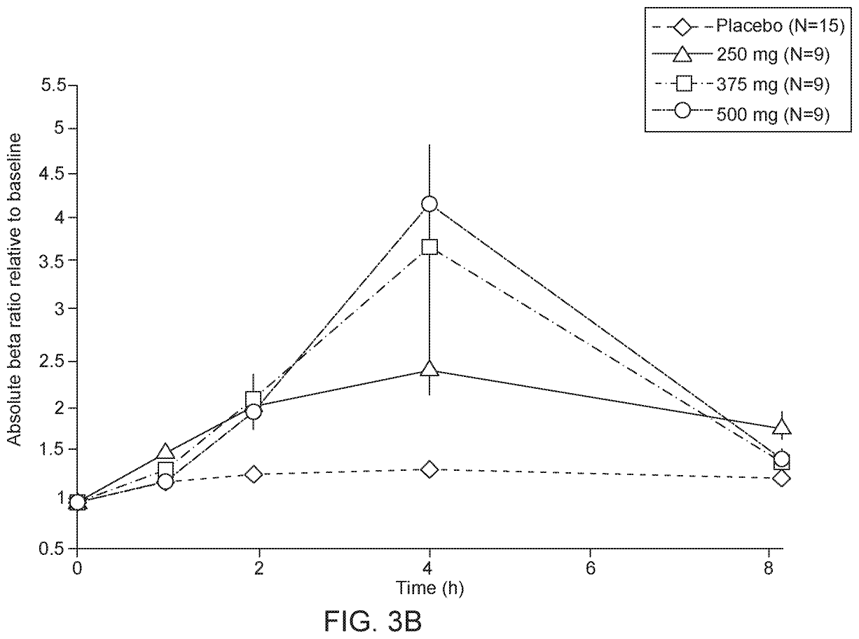

A maximum drop of saccade peak velocity was observed at hour 4 post-dose of day 1 across all doses tested as shown in FIG. 3A. The maximum effects for the video-oculography (VOG) assessments were observed around the Cmax, approximately hours 3-5 post-dose as demonstrated in FIG. 2A, which provides the concentration of allopregnanolone over time for subjects that underwent the VOG assessments.

The qEEG is a quantitative measurement of brain oscillations considering different frequency levels. Resting-state brain activity was recorded in 2 conditions (eyes closed and eyes open). For each electrode, a spectral analysis of the EEG signal was carried out to derive absolute and relative powers in standard frequency bands. The frequency ranges used are outlined in Table 9.

TABLE 9
Frequency Ranges for Spectral Analysis

| Nomenclature | Frequency range (Hz) |
|---|---|
| δ | 1.5 to <6.0 |
| θ | 6.0 to <8.5 |
| α1 | 8.5 to <10.5 |
| α2 | 10.5 to <12.5 |
| β1 | 12.5 to <18.5 |
| β2 | 18.5 to <21.0 |
| β3 | 21.0 to <30.0 |
| γ | 30.0 to <40.0 |

Compound 1 induced electroencephalogram modifications in all frequency bands studied, with the effects differing according to dose, time, and frequency band studied. The strongest effects were observed with the 500 mg dose, while 250 mg and 375 mg showed similar but reduced effects as to the 500 mg dose. FIG. 3B depicts the dose-dependent increase in power for the β-band (β1, β2, and β3) for cohorts 4 (250 mg), 5 (375 mg), and 6 (500 mg). The maximum increase in power is consistent with the observed Cmax (see FIG. 2A). In general, effects were higher after a single administration on day 1 as compared to day 7. Maximum effects were observed approximately 4 hours following administration on both day 1 and day 7.

### Pharmacokinetic Parameters and Assessments

PK parameters were computed from the individual plasma concentrations of Compound 1, combined allopregnanolone-containing molecules, and allopregnanolone. In addition, geometric mean was calculated for all PK parameters. Analyses using linear models were performed to assess dose proportionality.

The following PK parameters as described in Table 10 and Table 11 were estimated.

TABLE 10
PK Parameters for Part 1 and Part 2

| Parameter | Definition |
|---|---|
| Cₘₐₓ | Maximum concentration |
| tₘₐₓ | Time to maximum concentration |
| tₗₐₛₜ | Time to last measurable plasma concentration |
| AUC₀₋ₗₐₛₜ | Area under the plasma concentration-time curve (AUC) from time zero |
| | (time of dosing) to the last measurable concentration (AUC₀₋ₗₐₛₜ) |
| AUC_{0-inf} | AUC from time zero (from the start of infusion time) to infinity |
| t_{1/2} | Terminal elimination half-life |
| λz | Terminal elimination rate constant |
| CL/F | The apparent volume of distribution during the terminal phase |
| | (associated with λ_{z}) |
| V_{z}/F | The total body clearance of drug from plasma (volume × time−1) |

TABLE 11
PK Parameters for Part 3

| Parameter | Definition |
|---|---|
| Cₘₐₓ | Maximum concentration after first dose |
| Cₗₐₛₜ | Last measurable plasma concentration after first dose |
| tₘₐₓ | Time to maximum concentration after first dose |
| tₗₐₛₜ | Time to last measurable plasma concentration after first |
| | dose |
| AUC₍₀₋ₗₐₛₜ₎ | Area under the plasma concentration-time curve (AUC) |
| | from time zero (from the time of dosing) to the last |
| | measurable concentration (AUC₀₋ₗₐₛₜ) after first dose |
| AUC_{0-inf} | AUC from time zero (from the start of infusion time) to |
| | infinity after first dose |
| AUC_{0-τ} | AUC from time zero (from the start of infusion time) to τ |
| | after first dose |
| t_{1/2} | Terminal elimination half-life after first dose |
| CL/F | The apparent volume of distribution during the terminal |
| | phase (associated with λ_{z}) after first dose |
| V_{z}/F | The total body clearance of drug from plasma |
| | (volume x time-1) after first dose |
| C_{min,ss} | Observed minimum concentration in the dosing interval |
| | at steady state |
| C_{av,ss} | Average steady state concentration, using the following |
| | formula C_{av,ss} = AUC_{0-τ}/τ |
| | Where, τ = dosing interval |
| C_{trough,ss} | The quantifiable concentration at the end of the dosing |
| | interval at steady state |
| T_{max,ss} | Time to reach maximum concentration in the dosing |
| | interval at steady state. If the same Cₘₐₓ concentration |
| | occurs at different time points, Tₘₐₓ is assigned to the |
| | first occurrence of Cₘₐₓ |
| T_{min,ss} | Time of minimum observed concentration in the dosing |
| | interval at steady state |
| T_{lag} | Time of observation prior to the first observation with a |
| | measurable (non-zero) concentration |
| C_{24,ss} | The concentration observed at 24 hours post-dose at |
| | steady state |
| AUC_{0-t,ss} | Area under the drug concentration-time curve, from time |
| | zero (time of dosing) to the last measurable concentration |
| | at steady state using the ‘Linear Up and Log Down’ |
| | method |
| ${AUC}_{0 - τ , ss}^{a}$ | Area under the plasma concentration-time curve over a |
| | dosing interval at steady state using the ‘Linear Up and |
| | Log Down’ method |
| AUC_{0-12 hr,ss} | Area under the drug concentration-time curve, from time |
| | zero (time of dosing) to 12 hours post-dose at steady |
| | state using the ‘Linear Up and Log Down’ method |
| AUC_{0-24 hr,ss} | Area under the drug concentration-time curve, from time |
| | zero (time of dosing) to 24 hours post-dose at steady |
| | state using the ‘Linear Up and Log Down’ method |
| AUC_{0-inf ss} | Area under the drug concentration-time curve, from time |
| | zero (time of dosing) to infinity (∞) using the following |
| | formula |
| | ${AUC}_{0 - ∞} = {AUC}_{0 - t} + \frac{{C}_{t}^{′}}{{λ}_{z}}$ |
| | Where, C′ₜ is the observed concentration at the time t |
| | (last time point with a measurable plasma concentration |
| | above the quantification limit) at which quantification |
| | was still possible, the calculation of λ_{z} or kₑₗ is given |
| | below |
| AUC_{%extrap} | The percentage of the AUC that has been extrapolated |
| | beyond the last observed data point after first and |
| | multiple doses, using the following formula |
| | ${AUC}_{\% extrap} = ( \frac{{AUC}_{0 - ∞} - {AUC}_{0 - t}}{{AUC}_{0 - ∞}} ) * 100$ |
| λ_{z} or kₑₗ | The apparent terminal elimination rate constant will be |
| | estimated from a regression of ln (C) versus time over |
| | the terminal log-linear drug disposition portion of the |
| | concentration-time profiles |
| t_{1/2} | Apparent terminal half-life, using the following formula |
| | ${T}_{1 / 2} = \frac{Ln ( 2 )}{{λ}_{z}} .$ |
| CL/Fₛₛ | Apparent total plasma clearance at steady state, using the |
| | following formula |
| | CL/Fₛₛ = Dose/ AUC_{0-τ} |
| V_{z}/Fₛₛ | Apparent terminal volume of distribution at steady state, |
| | using the following formula |
| | CL/Fₛₛ = (Dose)/ (λ_{z} · AUC_{0-τ}) |
| MRT | Mean residence time, using the following formula |
| | $MRT = \frac{{AUC}_{0 - τ , ss} + τ ( {AUMC}_{0 - inf} - {AUC}_{0 - τ , ss} )}{{AUC}_{0 - τ , ss}}$ |
| | AUMC_{0-inf,ss} is the first moment of AUC_{0-inf,ss}. |
| Swing | Determined using the following formula |
| | Swing = (C_{max,ss} - C_{min,ss})/(C_{min,ss}) |
| Fluctuation | Determined using the following formula |
| % | Fluctuation % = 100 · (C_{max,ss} - C_{min,ss})/(C_{av,ss}) |
| R_{acc (Cmax)} | Accumulation ratio (Cₘₐₓ on Day 7/Cₘₐₓ on Day 1) |
| R_{acc (AUC)} | Accumulation ratio |
| | (AUC₀₋ₗₐₛₜ on Day 7/AUC₀₋ₗₐₛₜ on Day 1) |

### Safety and Tolerability

Compound 1 was generally well tolerated. There were no severe or serious drug-related adverse events. Most adverse events were mild, central nervous system (CNS)-related (Nervous System Disorder AEs), and dose-dependent. The most common adverse event was somnolence, which was most frequently observed at doses of Compound 1 greater than or equal to 500 mg. Food did not impact safety or tolerability of Compound 1. A summary of treatment-emergent adverse events for the MAD study cohorts (cohorts 4-8) is provided in Table 12.

TABLE 12
Summary of Treatment-Emergent Adverse Events for MAD Study

| | | | Cohort | Cohort | Cohort | Cohort | Cohort |
|---|---|---|---|---|---|---|---|
| | | | 4 | 5 | 6 | 7 | 8 |
| System Organ | Adverse | Pooled | 250 mg | 375 mg | 500 mg | 375 mg | 375 mg |
| Class | Event | Placebo | QAM | QAM | QAM | QHS | QHS |
| — | Any TEAE | 11 (73.3%) | 7 (77.8%) | 6 (66.7%) | 9 (100%) | 6 (66.7%) | 2 (22.2%) |
| Nervous | Somnolence | 10 (66.7%) | 7 (77.8%) | 6 (66.7%) | 9 (100%) | 5 (55.6%) | 1 (11.1%) |
| System | Balance | 0 | 0 | 0 | 7 (77.8%) | 2 | 0 |
| Disorders | Disorder | | | | | (22.2%) | |
| | Cognitive | 0 | 0 | 1 (11.1%) | 4 (44.4%) | 2 (22.2%) | 0 |
| | Disorder | | | | | | |
| | Headache | 0 | 0 | 0 | 0 | 1 (11.1%) | 0 |
| | Dizziness | 1 (6.7%) | 0 | 0 | 1 (11.1%) | 1 (11.1%) | 0 |
| | Sedation | 0 | 0 | 0 | 0 | 0 | 1 (11.1%) |
| | Disturbance | 0 | 0 | 0 | 1 (11.1%) | 1 (11.1%) | 0 |
| | in attention | | | | | | |
| | Lethargy | 0 | 0 | 0 | 1 (11.1%) | 0 | 0 |
| Psychiatric | Euphoric | 0 | 0 | 0 | 1 (11.1%) | 1 (11.1%) | 0 |
| Disorders | Mood | | | | | | |
| | Hallucination, | 0 | 0 | 0 | 1 (11.1%) | 0 | 0 |
| | Visual | | | | | | |
| | Abnormal | 0 | 0 | 1 (11.1%) | 0 | 0 | 0 |
| | Dreams | | | | | | |
| Gastrointestinal | Diarrhoea | 0 | 0 | 0 | 0 | 1 (11.1%) | 0 |
| Disorders | | | | | | | |
| General | Fatigue | 1 (6.7%) | 0 | 0 | 0 | 0 | 0 |
| Disorders and | | | | | | | |
| Administration | | | | | | | |
| Site Conditions | | | | | | | |
| Respiratory, | Rhinorrhoea | 1 (6.7%) | 0 | 0 | 0 | 0 | 0 |
| Thoracic, and | | | | | | | |
| Mediastinal | | | | | | | |
| Disorders | | | | | | | |

Participant disposition for the single ascending dose (SAD), food effect (FE), and multiple ascending dose (MAD) studies is summarized in Table 13.

TABLE 13
Participant Disposition

| | | Part 1: | | | Part 2: | Part 3: |
|---|---|---|---|---|---|---|
| | | SAD | | | FE | MAD |
| | | Cohort 1 | Cohort 2 | Cohort 2a | Cohort 3 | Cohorts 4-8 |
| Variable | Statistics | N = 11 | N = 9 | N = 10 | N = 9 | N = 60 |
| Age (years) | N | 11 | 9 | 10 | 9 | 60 |
| at Screening | Mean | 28.6 | 32 | 27.7 | 28.3 | 29.9 |
| | SD | 8.4 | 8.5 | 8.2 | 7 | 9 |
| | Median | 25 | 30 | 25.5 | 28 | 28 |
| | Minimum | 21 | 21 | 19 | 18 | 18 |
| | Maximum | 48 | 48 | 46 | 40 | 54 |
| Sex n (%) | Female | 4 (36.4%) | 2 (22.2%) | 1 (10.0%) | 2 (22.2%) | 24 (40.0%) |
| | Male | 7 (63.6%) | 7 (77.8%) | 9 (90.0%) | 7 (77.8%) | 36 (60.0%) |
| Childbearing | Yes | 4 (100%) | 1 (50.0%) | 1 (100%) | 2 (100%) | 23 (95.8%) |
| potential | | | | | | |
| n (%) | | | | | | |
| Ethnicity n | Hispanic/Latino | 3 (27.3%) | 1 (50.0%) | 1 (10.0%) | 2 (22.2%) | 6 (10.0%) |
| (%) | | | | | | |
| Race n (%) | Asian | 3 (27.3%) | 1 (11.1%) | 3 (30.0%) | 1 (11.1%) | 11 (18.3%) |
| | White | 6 (54.5%) | 8 (88.9%) | 7 (70.0%) | 7 (77.8%) | 36 (60.0%) |
| | Other | 2 (18.2%) | 0 | 0 | 1 (11.1%) | 7 (11.7%) |
| BMI (kg/m²) | Mean (SD) | 24.81 (2.05) | 23.64 (3.73) | 24.99 (2.18) | 26.12 (3.13) | 24.18 (3.15) |
| at Screening | | | | | | |

### Example 2: a Study to Assess the Effects of Compound 1 on the Stress Hormone Response in Human Subjects

A double-blind, placebo-controlled study was performed in 80 healthy participants to determine the effects of a single dose of Compound 1 in a validated and standardized behavioral challenge, the Trier Social Stress Test (TSST). The TSST is a reliable behavioral model used to examine the neurobiological response to acute stress in humans, whereby an acute stress (and associated acute stress response) is induced under experimentally controlled conditions. The TSST induces stress through public speaking. It incorporates social evaluation and unpredictability, by obliging the person to speak in front of an unresponsive audience and is further followed by a surprise mental arithmetic test. The TSST combines elements of a social-evaluative threat and uncontrollability to produce a consistent and robust physiological and psychological stress response in humans. On the day of the study, the participant was tasked with a speech prompt, for which they had 3 minutes to prepare. The participant was then required to present the speech for 5 minutes to a panel while under the pretense of being recorded. Following the speech, participants were given an unexpected mental arithmetic task. The TSST is more fully described in Allen A et al., Neurobiol. Stress (2017), February; 6:113-126.

The study included a validation cohort and a randomized cohort. The validation cohort (n=10) was given placebo for the purposes of validating the TSST and study endpoints. The randomized cohort (n=80) were randomized and stratified by sex to placebo or 375 mg of Compound 1 in a 1:1 ratio. The cohorts are described in Table 14. An overview of the TSST study design is also depicted in FIG. 4C.

TABLE 14
Cohorts for TSST Study

| | |
|---|---|
| Compound | Number of Subjects |

| Cohort | 1 Dose | Compound 1 | Placebo |
|---|---|---|---|
| Validation Cohort | Not Applicable | — | 10 |
| Randomized Cohort | 375 mg | 41 | 39 |

Compound 1 was formulated in combination with excipients Peceol™ (glyceryl monooleate), Kolliphor® RH40 (macrogolglycerol hydroxystearate) and super-refined sesame oil as a clear to yellow viscous solution. Each gram of formulation contains 300 mg of Compound 1, 189 mg of Peceol™, 336 mg Kolliphor® RH40, and 175 mg of super-refined sesame oil. To prepare the final dosage, the required quantity is manually filled into size 17.5 oblong capsules. The placebo contained vehicle only, i.e., the above formulation without Compound 1.

The objective of the study was to evaluate the efficacy of Compound 1 vs. placebo in blunting the response to endpoints measured during and after the TSST procedure. The primary efficacy endpoint was the change from baseline (before TSST procedure) to the peak (maximum change) in salivary cortisol levels as measured before, during, and after the TSST procedure. The primary outcome for determining this was salivary cortisol levels. The key secondary outcomes were be the Numerical Rating Scale (NRS) for stress, anxiety, fear, embarrassment, and excitement. Additional secondary outcomes were the State Trait Anxiety Index (STAI), and blood pressure. Safety and tolerability (i.e., incidence, severity, and duration of treatment-emergent adverse events (TEAEs), serious adverse events (SAEs), and incidence of other clinically relevant signs) were also monitored. Prior to the TSST, a post-dose sample was collected for pharmacokinetic analysis.

To normalize for food effects, participants were served a standardized meal to be consumed approximately 30 minutes prior to dosing with Compound 1. Compound 1 was administered with 240 mL of RT water ~3-4 hours prior to the start of the TSST.

### Study Endpoints and Outcomes

Pharmacodynamic and patient reported outcomes were collected prior, during, and after the TSST to assess the effectiveness of Compound 1 on blunting and/or stress response.

Cortisol levels (including, for example, salivary cortisol levels) are a robust and sensitive marker of stress and the stress hormone response. Salivary cortisol concentrations were collected and measured throughout the study, with the primary endpoint being the maximum change from baseline (pre-TSST).

The STAI-Y is a 40-item patient-completed questionnaire, with two subscales (20 items each), S-Anxiety and T-Anxiety. STAI-Y1 (State) was collected at baseline (pre-TSST), just prior to the start of the TSST procedure, and after completion of the TSST procedure. STAI-Y2 (Trait) was assessed at screening and baseline (pre-TSST). The S-Anxiety questionnaire assesses the intensity of current feelings in the moment, whereas the T-Anxiety questionnaire assesses frequency of feelings in general. The scores for each subtest are between 20 to 80, with higher scores indicative of greater anxiety levels.

A selection of study-specific numerical rating scales (NRS) which include questions for stress, anxiety, embarrassment, fear, and excitement were used. Questions were scored from 0 (not at all) to 10 (most imaginable).

Blood pressure was collected at pre-TSST, recovery, and before discharge.

A single post-dose PK sample was collected on Day 1 at 45 to 60 minutes prior to the TSST to confirm sufficient exposure to Compound 1.

Compound 1 achieved the study's primary endpoint. As shown in FIG. 4A, Compound 1 resulted in a partial blunting of increases in salivary cortisol levels as measured by a Least Squares Mean (LSMean) change from baseline (pre-TSST). The blunting of salivary cortisol levels was significantly different from placebo up to 30 minutes post-end of TSST. The Log 10 LSMean maximal change from baseline (pre-TSST) for salivary cortisol levels was significantly different for Compound 1 vs placebo (p-value=0.0001) (FIG. 4B). The maximum non-transformed mean showed a ~2.2× decrease in salivary cortisol levels versus placebo. Across all time points, the mean reduction in salivary cortisol levels post-TSST was 54.4% (ranging from 62.8% at 10 minutes post-TSST to 45.5% at 60 minutes post-TSST). The treatment effect size of Compound 1 versus placebo was 0.72, as measured by Cohen's d (a frequently utilized way to measure effect size and which is further described in Cohen J. (1988). Statistical Power Analysis for the Behavioral Sciences (2nd ed.)).

Compound 1 was well tolerated in the TSST study; all treatment related adverse effects (TEAEs) were transient, mild or moderate, and consistent with the known pharmacology profile of allopregnanolone. TEAEs that occurred at >5% were somnolence (29% in subjects receiving Compound 1 vs. 13% in placebo), dizziness (20% in subjects receiving Compound 1 vs 3% in placebo), and headache (7.3% in subjects receiving Compound 1 vs. 7.7% in placebo).

Participant disposition for the TSST study is summarized in Table 15.

TABLE 15
Participant Disposition for TSST Study

| | | Placebo | Compound 1 | Overall |
|---|---|---|---|---|
| Variable | Statistics | (N = 39) | (N = 41) | (N = 80) |
| Age (years) | n | 39 | 41 | 80 |
| at Screening | Mean (SD) | 30.2 (9.6) | 29.7 (10.4) | 30.0 (10.0) |
| | Minimum | 19 | 19 | 19 |
| | Maximum | 52 | 54 | 54 |
| Sex n (%) | Female | 8 (20.5%) | 10 (24.4%) | 18 (22.5%) |
| | Male | 31 (79.5%) | 31 (75.6%) | 62 (77.5%) |
| Ethnicity | Hispanic | 6 (15.4%) | 3 (7.3%) | 9 (11.3%) |
| n (%) | or Latino | | | |
| | Not Hispanic | 32 (82.1%) | 38 (92.7%) | 70 (87.5%) |
| | or Latino | | | |
| | Not Reported | 1 (2.6%) | 0 | 1 (1.3%) |
| Race | American Indian | 2 (5.1%) | 1 (2.4%) | 3 (3.8%) |
| n (%) | or Alaska Native | | | |
| | Asian | 6 (15.4%) | 10 (24.4%) | 16 (20.0%) |
| | Black or | 0 | 1 (2.4%) | 1 (1.3%) |
| | African | | | |
| | American | | | |
| | White | 30 (76.9%) | 29 (70.7%) | 59 (73.8%) |
| | Other | 1 (2.6%) | 0 | 1 (1.3%) |
| BMI (kg/m²) | n | 39 | 41 | 80 |
| at Screening | Mean | 24.51 (2.67) | 24.79 (2.37) | 24.65 (2.51) |
| | Minimum | 19.2 | 19.1 | 19.1 |
| | Maximum | 29.1 | 28.7 | 29.1 |

### Example 3: A Study to Assess the Effects of Compound 1 in Adults with Major Depressive Disorder (MDD) with or without Anxious Distress

A randomized, parallel-group, double-blind, placebo-controlled study will be performed to assess the efficacy, safety, and tolerability of Compound 1 in adults with major depressive disorder (MDD) with or without anxious distress.

Eligible subjects with MDD will be randomized in a 1:1 ratio to either Compound 1 or placebo cohorts within strata based on the presence or absence of the anxious distress specifier as determined at baseline. Approximately 50-60% of subjects will have MDD with anxious distress and approximately 40-50% of subjects will have MDD without anxious distress.

The duration of the study will be approximately 80 days, including screening (up to 4 weeks), treatment period (6 weeks), and post-treatment safety follow-up (1 week). Eligible participants who complete all visits will have the option to enter a 6-week open-label extension.

### Treatment and Dosing

An initial starting amount of 125 mg of Compound 1 or placebo will be administered once at bedtime (qHS) on study days 0-2. The dose of Compound 1 will be increased to 250 mg qHS on study days 3-6. Dose adjustment will not be permitted during the first week of study treatment—any subject requiring dose adjustment during the first week will be discontinued from study treatment.

Subjects will then receive Compound 1 at either 125 mg, 250 mg, or 375 mg or placebo through the end of the study treatment period, with the last dose taken during the evening of Study Day 41. During study days 7-28, dose adjustments will be permitted based on assessment of safety, tolerability, and effectiveness of study treatment. Participants who require dose adjustment after study day 28 will be discontinued from study treatment. An overview of the treatment and dosing schedule is provided in Table 16.

TABLE 16
Overview of Treatment and Dosing Schedule

| | Dosing Amount (orally administered |
|---|---|
| Study Day | once per day at bedtime (qHS) |
| Days 0-2 | 125 mg Compound 1 or placebo |
| Days 3-6 | 250 mg Compound 1 or placebo |
| Days 7-28 | 125, 250, or 375 mg Compound 1 or placebo |
| Days 29-41 | Dose amount attained at Day 28 |

Dose reduction may occur if the subject is experiencing intolerability associated with the current study dose regimen. Dose increase may occur if the subject is adequately tolerating the current study dose and may be experiencing suboptimal or inadequate treatment effect. The dose may be maintained without change if the subject is both adequately tolerating the current dose amount and experiencing satisfactory treatment response or outcome.

Following day 41, subjects may be administered 250 mg of Compound 1 (if they were receiving a lower or higher dose at day 41) or continue to receive 250 mg of Compound 1, administered qHS for an additional 6 weeks (42 days). Dose reduction to 125 mg of Compound 1 administered qHS is permitted throughout the 6-week period. Dose increase to 375 mg of Compound 1 administered qHS is permitted throughout the 6-week period.

### Efficacy Objectives and Endpoints

The primary objective will be to characterize the effects of Compound 1 as a monotherapy on depressive symptoms in subjects with MDD with anxious distress or MDD without anxious distress. The primary objective will be measured as a change from baseline to study day 42 in the Hamilton Depression Rating Scale-17 (HAM-D-17) total score.

The key secondary objective will be to characterize the effects of Compound 1 on overall illness severity in participants with MDD with anxious distress or MDD without anxious distress. The key secondary objective will be measured as a change from baseline to study day 42 in the clinician global impression-severity (CGI-S).

Additional secondary efficacy objectives will be to characterize the effects of Compound 1 on depression, anxiety, functioning, and quality of life in participants with MDD with anxious distress or MDD without anxious distress. The additional secondary efficacy objects will be measured as a change from baseline to study day 42 in the clinician global impression-improvement (CGI-S), Hamilton Anxiety Rating Scale (HAM-A), Sheehan Disability Scale (SDS), the Quality of Life, Enjoyment, and Satisfaction Questionnaire-Short Form (Q-LES-Q-SF), and the Overall Anxiety Severity and Impairment Scale (OASIS).

The HAM-D-17 is a clinician-administered rating scale designed to assess the severity of symptoms in subjects diagnosed with depression. The HAM-D-17 comprises individual ratings related to the following symptoms: depressed mood (sadness, hopeless, helpless, worthless), feelings of guilt, suicide, insomnia (early, middle, late), work and activities, retardation (slowness of thought and speech; impaired ability to concentrate; decreased motor activity), agitation, anxiety (psychic and somatic), somatic symptoms (gastrointestinal and general), genital symptoms, hypochondriasis, loss of weight, and insight.

The HAM-D-17 assessment is scored on a scale of 0-52, with higher scores indicating more severe depression. A score of 24-52 indicates severe depression, 17-23 moderate depression, 8-16 mild depression, and 0-7 normal, absence, or remission of depression. The total score is calculating by adding the scores from each question. An assessment time frame of the past 7 days (1 week) will be used at screening and baseline. For all other visits, the time frame will be since the previously recorded visit. The HAM-D-17 assessment is further described in Hamilton M “A rating Scale for Depression” J Neurol Neurosurg Psychiatry (1960) 23:56-62.

The Hamilton Anxiety Rating Scale (HAM-A) is a 14-item scale to rate the severity of symptoms of anxiety. Each of the 14 items is defined by a series of symptoms, and measures both psychic anxiety (mental agitation and psychological distress) and somatic anxiety (physical complaints related to anxiety). Scoring for HAM-A is calculated by assigning scores of 0 (not present) to 4 (very severe), with a total summed score range of 0-56. A score less than 17 indicates mild severity, 18-24 mild to moderate severity, and 25-30 moderate to severe severity.

The clinical global impression-severity (CGI-S) scale is a 7-point scale that requires the clinician to rate the severity of illness at the time of the assessment. Raters select one response based on the following question: “Considering your total clinical experience with this particular population, how ill is the patient at this time?” Scores are 1=normal, not at all ill; 2=borderline ill; 3=mildly ill; 4=moderately ill; 5=markedly ill; 6=severely ill; and 7=among the most extremely ill patients.

The clinical global impression-improvement (CGI-I) scale is a 7-point scale that requires the clinician to assess how much the subject's illness has improved or worsened relative to the baseline state at the beginning of the intervention. Raters select one response based on the following question: “Compared to your patient's condition at the beginning of treatment, how much has your patient changed?” Scores are 1=very much improved; 2=much improved; 3=minimally improved; 4=no change; 5=minimally worse; 6=much worse; and 7=very much worse.

The Sheehan Disability Scale (SDS) is a patient-reported outcome measure comprising a 5-item questionnaire that assesses functional impairment and associated disability. The first 3 items cover (1) work/school, (2) social life, and (3) family life/home responsibilities using a rating scale from 0-10. The SDS also has 1 item (4) assessing days lost from school or work and 1 item (5) assessing days of underproductivity. The score for the first 3 items are summed to create a total score of 0-30, with a higher score indicating greater impairment. The recall period is over 7 days.

The Quality of Life, Enjoyment, and Satisfaction Questionnaire-Short Form (Q-LES-Q-SF) assesses general activities using a 5 point scale ranging from 1 (very poor) to 5 (very good). A total score is derived from 14 items with a maximum score of 70, with higher scores indicating greater life satisfaction and enjoyment. Subjects will rate their satisfaction with the following domains of activity: physical health, feelings, work, household duties, school/course work, leisure time activities, and social relations.

The Overall Anxiety Severity and Impairment Scale (OASIS) is a self-report scale designed to assess the frequency and severity as well as impairment associated with anxiety conditions. The scale consists of 5 items each scored from 0-4 with a maximum total score of 20. Higher scores indicate greater anxiety-related severity and interference with functioning. A time frame of 7 days will be used.

The Patient Global Impressions Scale-Severity (PGI-S) is a patient reported counterpart to the CGI-S. The PGI-S is a single item, 6-point scale based on the CGI and adapted to the subject. The PGI-S assesses overall severity by the study subject over the past 7 days.

The Patient Global Impressions Scale-Change (PGI-C) is a patient reported counterpart to the CGI-I. The PGI-C is a single item 7-point scale indicating the level of overall improvement as assessed by the study subject. A time frame of the past 7 days compared to baseline will be used.

### Safety Objectives and Endpoints

The safety objective will be to evaluate the safety and tolerability of orally-administered Compound 1 in participants with MDD with anxious distress or MDD without anxious distress. Safety will be measured by the incidence, severity, and duration of treatment-emergent adverse events (TEAEs), serious adverse events (SAEs), the incidence of clinically significant vital signs, pulse oximetry, electrocardiograms (ECGs), and laboratory or physical examination findings, as well as the Columbia Suicide Severity Rating Scale (C-SSRS) and Physician Withdrawal Checklist-20 (PWC-20) will also be assessed.

Additionally, the change from baseline to study day 42 will be assessed for the Pittsburgh Sleep Quality Index (PSQI) and Epworth Sleepiness Scale (ESS), as well as the adverse childhood experiences questionnaire (ACE-Q).

The Epworth Sleepiness Scale (ESS) is composed of 8 items that assess the likelihood of falling asleep in real-world situations, such as reading, watching television, or driving. Each item is scored from 0-3 for a total score of 0-24, with higher scores indicating a greater severity of excessive daytime sleepiness.

The Pittsburgh Sleep Quality Index (PSQI) consists of 19 self-reported items belonging to 1 of 7 subcategories: subjective sleep quality, sleep latency, sleep duration, habitual sleep efficiency, sleep disturbances, use of sleep medication, and daytime dysfunction.

The Adverse Childhood Experiences Questionnaire (ACE-Q) is a 10-item self-reported measure that assesses adverse or traumatic experiences before the age of 18. These exposures may include past physical or sexual abuse, domestic violence, substance use, and incarceration. The ACE-Q is scored from 0-10, with higher scores indicating increased levels of adverse experiences.

Based on observed somnolence at higher doses (see Example 1), a driving restriction and hazardous activities/complex machinery restriction of at least 6 hours after dosing has been implemented.

### Pharmacokinetics Objectives and Endpoints

The population pharmacokinetic parameters of allopregnanolone in study subjects will be determined using population PK and exposure-response analysis of allopregnanolone in the population under study.

### Inclusion Criteria

Subjects will be male or female between 18 and 65 years of age, inclusive. Subjects must have a primary diagnosis of MDD. Subjects with a diagnosis of co-morbid generalized anxiety disorder (GAD), social anxiety disorder, or panic disorder (with or without agoraphobia) may be included if not the focus of treatment over the past 6 months prior to screening and that MDD is considered to be the primary diagnosis at screening and baseline.

Eligible subjects must have a current depressive episode of at least 4 weeks, but no greater than 18 months in duration prior to screening.

Subjects will have a HAM-D-17 score of greater than or equal to 23 at screening and baseline (prior to dosing). On day 1, subjects must have a HAM-D-17 score that is no more than a 20% improvement from the HAM-D-17 score at the time of screening. For example, if the HAM-D-17 score is 28 at screening, an eligible subject must have a day 1 HAM-D-17 score of at least 23.

### Exclusion Criteria

The following exclusion criteria will be applied.

Subjects will not be eligible with a history of, or current presentation consistent with, any depressive episode with psychotic or catatonic features, any bipolar manic, hypomanic or mixed episode, and substance-induced (e.g., anti-depressant-induced) manic, hypomanic/mixed episode, bipolar disorder, including history of bipolar depression, or current presentation consistent with bipolar depression, schizophrenia, schizoaffective, or other psychotic disorder, obsessive-compulsive disorder, and/or any persistent neurocognitive disorder.

Subjects with a diagnosis or treatment for attention deficit hyperactivity disorder (ADHD) within the last 5 years prior to screening.

Subjects with a diagnosis or treatment for an eating disorder, including bulimia or anorexia nervosa, within the 5 years prior to screening.

A history of treatment-resistant depression defined as 2 or more failed treatments of adequate dose and duration in the current depressive episode.

Post-traumatic stress disorder active within 3 years of screening.

Borderline or antisocial personality disorder or other disorder of sufficient severity that could interfere with participation in the study.

Psychiatric hospitalization within current depressive episode.

Evidence or history of clinically significant haematological, renal, endocrine, pulmonary, gastrointestinal, cardiovascular, hepatic, neurologic, or allergic disease (excluding seasonal allergies).

Previous history of intolerance or significant adverse events, including drug allergy to allopregnanolone or any components of Compound 1 formulation, including sesame seeds or sesame seed products.

Any condition at screening or baseline, such as chronic diarrhea, inflammatory bowel disease, or prior surgery of the GI tract, that would possibly interfere with drug absorption, or any disease or condition that is likely to affect drug metabolism or secretion.

Having receiving any prohibited medications, supplements, or herbal products, including any anti-psychotic, anti-convulsant, anxiolytic, benzodiazepine, or anti-depressant treatment within 2 weeks or 5 half-lives of the medication, whichever is longer, prior to baseline.

History of electroconvulsive therapy, vagus nerve stimulation, transcranial magnetic stimulation, or any experimental CNS treatment during the current episode or in 6 months before screening (whichever is longer).

Hypo- or hyperthyroidism, unless stabilized on appropriate pharmacotherapy with no change in dose for at least 1 month before study start. Serum thyroid stimulating hormone must be greater than 0.75× the lower limit of normal and less than 1.25× the upper limit of normal at screening.

Current laboratory evidence, signs, or symptoms of hepatic or renal insufficiency.

Subjects with other abnormal laboratory test results, vital sign results, or ECG findings unless considered not medically significant by medical professionals.

Symptoms of dysphagia at screening or baseline or known difficulty in swallowing capsules.

Night-time shift work or other conditions that may be disruptive to a normal sleep/wake cycle.

Subjects with a positive result for human immunodeficiency virus (HIV) antibodies, hepatitis B antigen (HbsAg), or hepatitis C virus (HCV) antibodies at screening.

Subjects with a positive result for COVID-19 within 14 days prior to screening.

Malignancy or a history of malignancy with the exception of adequately treated or excised non-metastatic basal cell or squamous cell cancer of the skin or adequately treated cervical carcinoma-in-situ.

A history of drug or alcohol use disorder according to Diagnostic and Statistical Manual of Mental Disorders, Fifth Edition (DSM-5) criteria within 12 months before screening, or positive test result(s) for drugs of abuse or drugs with the potential for abuse, including barbiturates, opiates/opioids, phencyclidine, cocaine, cannabinoids, amphetamines, and benzodiazepines at screening. Subjects with a positive test for cannabinoids who do not meet DSM-5 criteria for moderate or severe substance abuse within 12 months before screening may be eligible.

Clinically significant risk of suicide or harm to self or others.

Treatment with an investigational product within 60 days or 5 half-lives (whichever is longer) preceding the first dose of study treatment, with no more than 2 prior investigational studies in the last year. A lifetime history of greater than 5 investigational studies is exclusionary.

Screening 12-lead ECG following at least 5 minutes of supine rest demonstrating a Fridericia corrected QT (QTcF) interval >450 msec (for males) or >470 msec (for females) or a QRS interval >120 msec.

Hypertension as defined by a supine blood pressure greater than or equal to 160 mmHg (systolic) or greater than or equal to 100 mmHg (diastolic) at screening.

Serum creatinine levels about the ULN at screening or an estimated Glomerular Filtration Rate value less than or equal to 80 mL/min.

Aspartate aminotransferase or alanine aminotransferase values greater than or equal to 1.5× the ULN.

Total bilirubin levels greater than or equal to 1.5×ULN except for subjects with Gilbert's syndrome.

The following concomitant medications, defined as any medication the subject takes after initial study treatment administration, are prohibited: all antidepressants, anxiolytics, mood stabilizers, antipsychotics, sedative hypnotics (including benzodiazepines), sedating antihistamines, opiates, anticonvulsants, lipase inhibitors, S-adenosyl methionine, St. John's wort, ephedra, kava kava, and neuromodulation or other medical devices intended to treat neuropsychiatric disorders.

Non-benzodiazepine sleep aids, including zolpidem 5-10 mg at bedtime, zolpidem controlled release 6.25-12.5 mg at bedtime, zaleplon 5-10 mg at bedtime, eszopiclone 1-2 mg at bedtime, melatonin 1-5 mg at bedtime, and ramelteon 8 mg at bedtime are allowed on an as needed basis.

We claim:

1. A method of treating a human subject having major depressive disorder (MDD), the method comprising orally administering to the human subject in need thereof a dose of Compound 1:

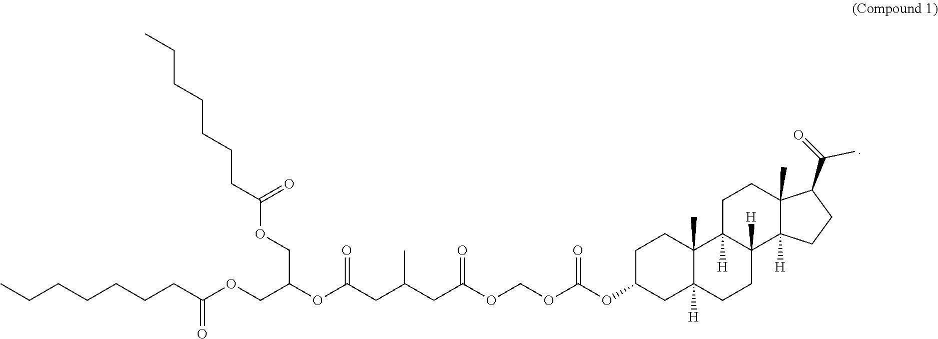

2. The method of claim 1, wherein the dose of Compound 1 is between 70-500 mg.

3. The method of claim 2, wherein the dose of Compound 1 is 70 mg, 125 mg, 140 mg, 250 mg, 280 mg, 375 mg, 420 mg, or 500 mg.

4. The method of any one of claims 1-3, wherein the dose of Compound 1 is 125 mg.

5. The method of any one of claims 1-3, wherein the dose of Compound 1 is 250 mg.

6. The method of any one of claims 1-3, wherein the dose of Compound 1 is 375 mg.

7. The method of any one of claims 1-6, wherein the human subject having major depressive disorder (MDD) has MDD with anxious distress.

8. The method of any one of claims 1-6, wherein the human subject having major depressive disorder (MDD) has MDD without anxious distress.

9. The method of any one of claims 1-8, wherein the dose of Compound 1 is administered once per day at bedtime (qHS).

10. A method of treating a human subject having major depressive disorder (MDD), the method comprising orally administering to the human subject in need thereof:
i) a daily initiation dose of Compound 1 for an initiation period of up to 7 days; and
ii) a daily maintenance dose of Compound 1 that begins after the initiation period; and wherein Compound 1 is:

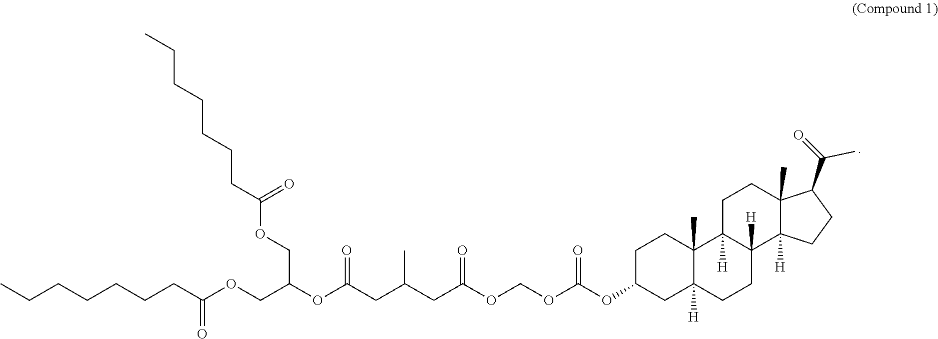

11. The method of claim 10, wherein the daily initiation dose of Compound 1 is 125 mg.

12. The method of claim 10, wherein the daily initiation dose of Compound 1 is 250 mg.

13. The method of any one of claims 10-12, wherein the daily maintenance dose of Compound 1 is 125 mg, 250 mg, or 375 mg.

14. The method of any one of claims 10-13, wherein the initiation period is 3 to 7 days.

15. The method of any one of claims 10-14, wherein the daily maintenance dose of Compound 1 is greater than the daily initiation dose of Compound 1.

16. The method of any one of claims 10-14, wherein the daily maintenance dose of Compound 1 is the same as the daily initiation dose of Compound 1.

17. The method of any of claims 10 and 12-14, wherein the daily maintenance dose of Compound 1 is smaller than the daily initiation dose of Compound 1.

18. The method of any one of claims 10-17, wherein the human subject having major depressive disorder (MDD) has MDD with anxious distress.

19. The method of any one of claims 10-17, wherein the human subject having major depressive disorder (MDD) has MDD without anxious distress.

20. The method of any one of claims 10-19, wherein the daily initiation dose of Compound 1 is administered once per day at bedtime (qHS).

21. The method of any one of claims 10-20, wherein the daily maintenance dose of Compound 1 is administered once per day at bedtime (qHS).
